(19) United States
(12) Patent Application Publication
(10) Pub. No.: US 2022/0389384 A1
(43) Pub. Date: Dec. 8, 2022

(54) PROCESS FOR ISOLATING NUCLEATED CELLS AND NUCLEATED CELL POPULATIONS AND USES THEREOF

(71) Applicant: MESOTEX, INC., GREAT RIVER, NY (US)

(72) Inventor: Giammaria SITAR, Lavagna (IT)

(73) Assignee: MESOTEX, INC., GREAT RIVER, NY (US)

(21) Appl. No.: 17/575,934

(22) Filed: Jan. 14, 2022

Related U.S. Application Data
(63) Continuation of application No. 16/091,722, filed on Oct. 5, 2018, now abandoned.
(60) Provisional application No. 62/319,523, filed on Apr. 7, 2016.

(51) Int. Cl. C12N 5/0775; C12N 5/00; A61K 35/28; C12Q 1/6883

(52) U.S. Cl. CPC C12N 5/0662; C12N 5/0087; A61K 35/28; C12Q 1/6883; C12Q 2600/156

(57) ABSTRACT

The present disclosure provides processes for isolating target nucleated cells, such as fetal mesenchymal stem cells, from non-nucleated red blood cells, populations of cells obtainable by the processes of the disclosure, and methods of using isolated targeted nucleated cells methods and their progeny for detecting fetal abnormalities and stem cell therapy.

### 1. CROSS REFERENCE TO RELATED APPLICATIONS

This application claims the priority benefit of U.S. provisional application No. 62/319,523, filed Apr. 7, 2016, the contents of which are incorporated herein in their entireties by reference thereto.

### 2. BACKGROUND

A major impediment to the recovery of nucleated cells (NCs) circulating in peripheral blood, e.g., fetal cells circulating in maternal blood, cancer cells circulating in the blood of patients in clinical remission, stem cells, endothelial cells and/or other very rare cells, is the very low total number of these cells in a single collection of blood.

The first challenged to obtaining NCs is that non-nucleated red blood cells (RBCs) outnumber the highly desirable NCs in both peripheral and non-peripheral blood. For example, fetal cells number 2-6 per 1 ml maternal blood, which is a ratio 1 fetal cell in 1-3 million nucleated maternal cells and 1 fetal cell in 1-2 billion red blood cells. Krabchi et al., 2001, Clin. Genet. 60:145-150. In non-peripheral blood such as umbilical cord blood (UCB), the ratio of NCs to RBCs is approximately 1000 to 1.

Many procedures have been devised with the aim of obtaining rare NCs maternal blood. However, finding a rare NC in the overwhelming amount of cells present in blood has been challenging, particularly in a manner that preserves their functionality.

The first step in most NC enrichment procedures entails depletion of RBCs from the blood sample containing the NCs. Commonly, in order to reduce the number of RBCs and plasma volume in either closed or open systems, methods involving manual or semi-automated centrifugation with or without the addition of exogenous media, such as ficoll, percoll, hydroxyethyl starch (HES), dextran, poligeline, and gelatin, are performed. However, these methods differ greatly in terms of final product volume, residual RBCs, cell viability, and the recovery of NCs, mononuclear cells (MNCs), CD34+ cells, colony forming units cells (CFUs), and long-term culture-initiating cells (LTC-ICs), not to mention the complexity of procedures and processing time required. See Tsang et al., 2001, Transfusion 41:344-352; Pilar Solves et al., 2005, Transfusion 45:867-873.

The classic and simplest method for separating non-nucleated cells from nucleated cells is by density gradient centrifugation. Density gradient centrifugation separates cells on the basis of cell density. A blood sample can be subjected to density gradient centrifugation using a density gradient material like Ficoll, Ficoll-Hypaque, Histopaque, Nycodenz and Polymorphprep, which are all solutions containing a red-blood-cells aggregating agent. After centrifugation, the peripheral blood sample forms a supernatant layer, which contains plasma and platelets; a nucleated cell layer at the interface between blood sample and the separation medium; and an agglutinated pellet at the bottom of the centrifugation tube which contains non-nucleated erythrocytes and some nucleated cells. The nucleated cell layer can be separated from the other layers, to produce a nucleated cell enriched sample from which non-nucleated cells have been largely removed. However, as in other procedures, NCs are often lost at unacceptably high rates in density gradient centrifugation.

Following RBC removal, NC enrichment process currently in use include discontinuous density gradient centrifugation, fluorescent activated cell sorting (FAGS), magnetic activated cell sorting (MACS), charge flow separation, micromanipulation, avidin-biotin columns magnetic ferro-fluids. Comparative analysis of these different procedures has been the subject of several reviews (Ho et al., 2003, Ann. Acad. Med Singap. 32:560-597; McEwan, 2005, Maternal Medicine Review 16:151-177; Kavanagh et al., 2010, Journal of Chromatography B, 878:1905-1911). Although various fetal cell types have been isolated from maternal blood such as trophoblasts, leukocytes, nucleated erythrocytes, platelets and haemopoietic progenitors, reliable and reproducible isolation of most types of fetal cells, or combinations of fetal cells, present in maternal blood seems an elusive goal.

Thus, there is a need in the art for a simple and reliable, preferably high yield method for both removing RBCs from a blood sample while retaining substantially all NCs and subsequently enriching target NCs to high purity in a reproducible manner. There is also a need for co-isolation/co-enrichment of at least two different types NCs, e.g., fetal cell types from one sample, e.g., fetal nucleated red blood cells and fetal mesenchymal stem cells. In certain cases, there is also a need in the art for a method that ensures minimal loss of function of the NCs.

### 3. SUMMARY

The present disclosure provides processes for isolating populations of target nucleated cells, particularly mesenchymal stem cells (MSCs), nucleated red blood cells, and CD34+ stem cells from non-nucleated red blood cells present in samples containing both the target nucleated cells and non-nucleated red blood cells. The processes comprise subjecting the sample to at least one of negative selection for the target cells, positive selection for the target cells, and density gradient centrifugation. Exemplary processes are described in Section 5.2 and embodiments 1 to 54 below.

The present disclosure also provides populations of target nucleated cells that are obtainable by the processes of the disclosure. In some embodiments, the populations comprise one, two, or all three of fetal mesenchymal stem cells (MSCs), fetal nucleated red blood cells (NRBCs), and fetal CD34+ stem cells. Exemplary cell populations are described in Section 5.3 and embodiments 55 to 57 below.

The present disclosure further provides methods of using the populations of the disclosure and one or more cells from the populations to detect fetal abnormalities. Exemplary diagnostic methods and uses of the populations and cells are described in Section 5.4.1 and embodiments 58 to 69 below.

The present disclosure further provides therapeutic uses for the populations of target nucleated cells of the disclosure. Exemplary therapeutic uses are described in Section 5.4.2 and embodiments 70 to 79 below.

### 4. BRIEF DESCRIPTION OF THE FIGURES

FIG. 1: a schematic view of an exemplary separation device.

FIG. 2A-2B: fetal nucleated cells isolated from maternal blood after three days (FIG. 2A) and one week (FIG. 2B) of in vitro expansion.

FIG. 3: Oct4 (1), Nanog (2), Sox2 (3), and GAPDH (4) amplification products from RT-PCR performed using cells shown in FIG. 2B.

FIG. 4A-4C: fetal mesenchymal stem cells imaged after 3 days (FIG. 4A), 6 days (FIG. 4B) and 9 days (FIG. 4C) in culture.

FIG. 5: fMSCs analyzed by fluorescence in-situ hybridization (FISH) using X and Y chromosomal hybridization probes and counterstained with DAPI.

### 5. DETAILED DESCRIPTION

### 5.1 Definitions

An aggregating agent is an agent that promotes the aggregation of non-nucleated red blood cells in a composition comprising non-nucleated red blood cells.

An antibody is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also any antigen binding fragment thereof (i.e., “antigen-binding portion”) or single chain thereof, fusion proteins comprising an antibody, and any other modified configuration of the immunoglobulin molecule that comprises an antigen recognition site, including, for example without limitation, single chain (scFv) and domain antibodies (e.g., human, camelid, or shark domain antibodies), maxibodies, minibodies, intrabodies, diabodies, triabodies, tetrabodies, vNAR and bis-scFv (see e.g., Hollinger and Hudson, 2005, Nature Biotech 23:1126-1136). An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA, and IgA₂. “Antibody” also encompasses any of each of the foregoing antibody/immunoglobulin types that has been modified to facilitate sorting and detection.

Antigen binding portion of an antibody, as used herein, refers to one or more fragments of an intact antibody that retain the ability to specifically bind to a given antigen (e.g., target X). Antigen binding functions of an antibody can be performed by fragments of an intact antibody.

A blood derived nucleated cell enriched fraction, as used herein, refers to a composition comprising nucleated cells from a sample of blood but containing no more than 20% of the non-nucleated red blood cells from the sample of blood. The term “blood derived nucleated cell enriched fraction” encompasses compositions that result from enrichment of a blood derived nucleated cell enriched fraction for a target nucleated cell type.

A blood fraction, as used herein, is a composition that comprises some, but not all, components of whole blood and can comprise a non-blood component, such as a buffer or cell culture media.

Compete, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present disclosure.

Heavy liquid when used in a process for separating a nucleated cell enriched fraction and a non-nucleated red blood cell enriched fraction from a mixture comprising nucleated cells, non-nucleated red blood cells, and an aggregating agent means a liquid having a density that is greater than the density of the liquid in the mixture comprising nucleated cells, non-nucleated red blood cells, and an aggregating agent.

Hematocrit, in reference to a sample containing non-nucleated red blood cells, refers to the ratio of the volume of non-nucleated red blood cells to a given volume of a sample. A typical sample is a mixture containing the non-nucleated red blood cells and other cell types, e.g., blood.

Negative selection refers to depletion of cells other than a target cell of interest from mixed cell population. Negative selection can be based on a marker that is absent from (or undetectable in or on) the target cell. Negative selection can also be based on other criteria, e.g., size, morphology, or other physical characteristics.

Negative immunoselection refers to depletion of cells utilizing an antibody, e.g., an antibody that selectively binds to one or more cell types other than the target cells of interest but does not specifically bind to the target cells.

A negative immunoselective antibody is an antibody that can be used in negative immunoselection, e.g., is an antibody that binds to a marker that is present on or in one or more cell types other than the target cells but is absent from the target cell. The antibody can bind to a marker on the cell surface or an internal marker, but the marker is preferably a surface marker to avoid the need for cell fixation.

Positive selection refers to selection of cells (e.g., for enrichment and/or isolation purposes) containing a target cell of interest from a mixed cell population. Positive selection can be based on a marker that is present on or in the target cell. In some embodiments, the marker absent from (or undetectable in or on) one or more cell types (other than the target cell) in the population (e.g., biological sample) from which the target cell is to be isolated or enriched (for example, maternal blood or a fraction of maternal blood when the target cell is an fNRBC). In further embodiments, the marker is absent from (or undetectable in or on) any cell type other than the target cell of interest in the population from which the target cell is to be isolated or enriched. Positive selection can also be based on other criteria, e.g., size, morphology, or other physical characteristics (e.g., adhesion to plastic).

Positive immunoselection refers to selection of cells utilizing an antibody, e.g., an antibody that binds to a marker that is present on or in the target cell of interest and which is therefore useful for positive selection.

A positive immunoselective antibody is an antibody that can be used in positive immunoselection, e.g., is an antibody that binds to a marker that is present on or in the target cell. In some embodiments, the antibody selectively binds to the target cell but does not specifically bind to one or more other cell types that may be present in a population of cells in which the target cell is present. The antibody can bind to a marker on the cell surface or an internal marker, but the marker is preferably a surface marker to avoid the need for cell fixation.

Selective binding with respect to a particular cell refers to the specific or preferential binding of an antibody to a marker present in or on at least one cell type in a mixed cell population (e.g., a nucleated cell enriched fraction) but absent from (or undetectable in or on) at least one other cell type in the population. By way of example, if in a mixed cell population containing cell types A, B, C, D, and E, an antibody only specifically binds to cell type A or cell types A and E, the antibody is said to selectively bind to cell types A or cell types A and E, respectively.

An antibody specifically binds or preferentially binds to a target if it binds with greater affinity, avidity, more readily, and/or with greater duration than it binds to other substances. For example, an antibody that specifically or preferentially binds to a marker present on fNRBCs is an antibody that binds this marker with greater affinity, avidity, more readily, and/or with greater duration than it binds to other markers. Specific binding or preferential binding does not necessarily require (although it can include) exclusive binding. Generally, but not necessarily, reference to “binding” means preferential binding.

### 5.2 Processes for Obtaining Populations of Target Nucleated Cells

This disclosure provides processes for obtaining populations of target nucleated cells, particularly mesenchymal stem cells (MSCs), nucleated red blood cells, and CD34° stem cells from mixtures containing them (e.g., blood, such as maternal peripheral blood).

In one aspect, the disclosure provides a process for isolating a population of target nucleated cells from non-nucleated red blood cells present in a blood derived nucleated cell enriched fraction containing no more than 20%, no more than 15%, no more than 10%, no more than 5%, or no more than 1% of the non-nucleated red blood cells from the blood, comprising subjecting the nucleated cell enriched fraction to one, two, or all three of negative selection for the target nucleated cells, positive selection for the target nucleated cells, and density gradient centrifugation. Processes for obtaining blood derived nucleated cell enriched fractions are described in Section 5.2.1. Negative selection methods useful in the processes of the disclosure are described in Section 5.2.2.1, positive selection methods useful in processes of the disclosure are described in 5.2.2.2, immunoselection techniques that can be used to perform negative and positive selection are described in Section 5.2.2.3, and density gradient separation methods that can be used in the processes of the disclosure are described in 5.2.2.3.

### 5.2.1 Bulk Separation of Nucleated Cells from Non-Nucleated Red Blood Cells

Blood derived nucleated cell enriched fractions can be obtained by a gravity sedimentation process in which nucleated cells are separated from most (e.g., 80% or more) of the non-nucleated red blood cells in a mixture comprising the nucleated cells and non-nucleated red blood cells (e.g., blood).

In one aspect, the mixture can be formed by combining a sample comprising nucleated cells and non-nucleated red blood cells with an aggregating agent or a solution comprising an aggregating agent. The aggregating agent promotes formation of red blood cell aggregates, known as rouleaux, which have a greater sedimentation rate than nucleated cells. Exemplary aggregating agents are described in Section 5.2.1.2, below. Without being bound by theory, it is believed that as the rouleaux sediment under the force of local gravity in the lumen of a sedimentation device, they displace a RBC depleted phase upward in the lumen, forming a lower layer enriched in RBCs and an upper phase enriched in nucleated cells. It is further believed, again without being bound by theory, that liquid that is displaced upward as the rouleaux sediment will pull along the slower settling nucleated cells, facilitating separation of the mixture into a nucleated cell enriched fraction and a non-nucleated nucleated red blood cell enriched fraction. However, nucleated cells can become entrapped within the aggregating RBC while they form rouleaux and in the rouleaux as they sediment, significantly reducing the yield of nucleated cells in the nucleated cell enriched fraction. Significant loss of nucleated cells can be acceptable in some instances, for example, in the field of transfusion hematology where large volumes of donor blood are available, but is unacceptable when the amount of sample is limited and/or the sample contains a rare cell type of interest, e.g., a fetal cell or a stem cell.

The separation processes described herein solve this problem by separating nucleated from non-nucleated cells in the lumen of a container under conditions that allow rouleaux to sediment more quickly than in traditional sedimentation processes. It was discovered that nucleated cell yield is significantly increased when separating the mixture in the lumen of a container sized so that the mixture has a height in the lumen during separation that is reduced as compared to traditional sedimentation methods.

As rouleaux sediment in a mixture comprising nucleated cells, non-nucleated red blood cells, and an aggregating agent, the density of the mixture increases toward the bottom of the lumen because density is hematocrit dependent. The sedimentation velocity of the rouleaux decreases as density increases. It is believed that in traditional sedimentation methods, the upward flow of liquid (plasma) caused by sedimenting rouleaux becomes insufficient to pull nucleated cells out of the sedimenting rouleaux as the sedimentation velocity of the rouleaux decreases. It is believed, again without being bound by any theory, that when the height of the mixture is reduced as compared to traditional sedimentation methods, the rate of upward liquid flow caused by sedimenting rouleaux exceeds the sedimentation velocity of the nucleated cells for a greater portion of the separation time, allowing for a greater number of nucleated cells to be pulled away from the sedimenting rouleaux. In some embodiments, the average height of the mixture in the lumen is less than 4 cm, less than 3.5 cm, less than 3 cm, less than 2 cm, less than 1.5 cm, or less than 1 cm. In some embodiments, the average height of the mixture in the lumen is 1 cm, 1.5 cm, 2 cm, 2.5 cm, 3 cm, 3.5 cm, or 4 cm. In other embodiments, the average height of the mixture in the lumen is in a range between any pair of the foregoing values, such as 1-4 cm, 1-3 cm, 1.5-2.5 cm, 2-3.5 cm, or 1-2 cm. Preferably, the average height of the mixture in the lumen is 1.5-2 cm.

### 5.2.1.1 Mixtures Comprising Nucleated Cells and Non-Nucleated Red Blood Cells

The mixture separated into a nucleated cell enriched fraction and a non-nucleated cell enriched fraction comprises nucleated cells, non-nucleated red blood cells, and one or more aggregating agents. In some embodiments, the mixture is obtained by combining a sample comprising the nucleated cells and non-nucleated red blood cells with an aggregating agent or a solution comprising the aggregating agent.

The separation processes described herein can be performed to separate nucleated cells from non-nucleated cells in blood. The blood can be, for example, peripheral blood (e.g., a peripheral blood sample obtained from a pregnant female, a subject afflicted with a cancer, or a healthy subject) or umbilical cord blood (UCB). The blood can be from any mammalian source, e.g., a domesticated animal (such as a cat or dog), livestock (e.g., cattle), a research animal (e.g., a mouse, rat or chimpanzee), and is most preferably human.

The blood can be whole blood (i.e., blood drawn directly from a subject) or processed blood. Processed blood can be whole blood diluted with an aqueous solution or a blood fraction. In some embodiments, the sample is a blood fraction that has been processed to remove some or all plasma. For example, plasma can be removed from whole blood by centrifuging whole blood to form a pellet containing nucleated cells and non-nucleated red blood cells and removing some or all of the supernatant, which comprises plasma. The pellet can then be resuspended in an aqueous solution to provide a blood fraction that is then separated according to a process of the disclosure. In some embodiments, a blood fraction is prepared by diluting blood with an aqueous solution, centrifuging the diluted blood to form a cell pellet containing nucleated cells and non-nucleated red blood cells, and resuspending the cell pellet in an aqueous solution after removing some of the plasma to provide a blood fraction containing nucleated cells, non-nucleated red blood cells and plasma. In some embodiments, the blood fraction contains at least 5%, at least 10%, at least 20%, at least 30%, at least 40%, at least 50%, or more than 50% of the plasma present in the whole blood used to make the blood fraction. In some embodiments, the blood fraction contains 5-10%, 10-20%, 20-30%, 20-50%, or 50-100% of the plasma present in the whole blood used to make the blood fraction, or any other range bounded by lower and upper limits selected from 5%, 10%, 20%, 30%, 40%, 50%, 60%, 70%, 80%, 90%, and 100%.

Aqueous solutions suitable for use in the processes of the disclosure (e.g., for diluting a sample comprising nucleated cells and non-nucleated red blood cells, preparing a blood fraction, and/or preparing a solution of an aggregating agent) include physiological solutions, i.e., solutions that have a similar pH and osmolarity and/or tonicity as blood, such as tissue culture media. Exemplary physiological solutions include Roswell Park Memorial Institute (RPMi) medium, Dulbecco's Phosphate Buffered Saline, Kreb's-Ringer Biocarbonate Buffer, Puck's Saline, Earle medium, and Hanks balanced salt solution. Plasma and mixtures of plasma and a second physiological solution can also be used as aqueous solutions in the processes of the disclosure.

The processes of this disclosure are particularly suited for separating rare nucleated cells from blood, such as stem cells or circulating cancer cells from adult peripheral blood and fetal cells from peripheral blood of a pregnant woman, into a nucleated cell enriched fraction that contains most of the rare nucleated cells and a non-nucleated red blood cell enriched fraction that contains most of the non-nucleated red blood cells and few, if any, of the rare nucleated cells. For diagnostic testing, the peripheral blood sample is typically 25-30 mL, particularly from pregnant women to ensure that the fetus is not harmed by the reduced maternal blood volume. The processes of the disclosure also permit improved yield of nucleated cells of interest from samples in which they are more prevalent, such as stem cells umbilical cord blood. The amount of blood obtainable from an umbilical cord is variable, and was found to range from 72 to 275 mL in one recent study. Nunes et al., 2015, Brazilian Journal of Hematology and Hemotherapy 37(1):38-42. The processes of the disclosure can be performed using all or part of a peripheral blood or umbilical cord blood sample, e.g., 10-20 mL, 20-30 mL, 20-50 mL, 50-100 mL, 100-150 mL, or more than 150 mL, if available. The amount of blood used can be selected based on the amount of blood available and the number and/or the type of nucleated cells of interest.

The volume of the mixture separated in the lumen of the container can vary based upon the type of sample used to form the mixture. For example, the volume of a mixture prepared from 25 mL of peripheral blood obtained from a pregnant woman can be one quarter of the volume of a mixture prepared from 100 mL of umbilical cord blood if prepared by the same process. In some embodiments, the volume of the mixture is less than 500 mL, less than 400 mL, less than 300 mL, less than 200 mL, less than 100 mL, less than 75 mL, less than 50 mL, less than 40 mL, less than 30 mL, or less than 25 mL. In some embodiments, the volume of the mixture is 25 mL to 50 mL, 50 mL to 100 mL, 100 mL to 200 mL, or 200 mL to 400 mL.

The amount of time necessary to separate the mixture into a nucleated cell enriched fraction and non-nucleated cell enriched fraction is dependent upon density and height of the mixture, and can be empirically determined by those skilled in the art. The density and height of the mixture are preferably selected so that separation of the mixture into a nucleated cell enriched fraction and a non-nucleated red blood cell enriched fraction is substantially complete in 2 to 15 minutes or even longer. In various embodiments, the separation is complete in 2 to 10 minutes, 2 to 5 minutes, 3 to 6 minutes, 4 to 12 minutes, 5 to 10 minutes, 2 to 8 minutes, 4 to 10 minutes, 3 to 7 minutes, 6 to 10 minutes, 5 to 8 minutes, or any other range bounded by lower and upper limits selected from 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes and 15 minutes.

Density of the mixture can be modulated by adjusting hematocrit of the mixture and by the use of aqueous solutions of different densities. Low density mixtures provide faster sedimentation of rouleaux and a greater upward pull of nucleated cells relative to high density mixtures. Hematocrit of the mixture can be modulated, for example, by adjusting hematocrit of the sample comprising the nucleated cells and non-nucleated cells prior to forming the mixture, adjusting the concentration of aggregating agent in the solution of aggregating agent so that more or less of the aggregating agent solution is needed, adding an amount of an aqueous solution to the mixture, or a combination thereof. In some embodiments, the mixture has a hematocrit value that is lower than the hematocrit value of whole blood, e.g., a hematocrit value that is one half of the hematocrit value of whole blood. In some embodiments, the hematocrit of the mixture, measured as the volume percentage of non-nucleated red blood cells in the mixture, is 10-45%, 10-30%, 10-20%, 15-45%, 15-30%, 15-20%, 20-45%, 20-30%, 25%-45%, 20-30%, 5%, 10%, 15%, 20%, 25%, 30%, 35%, 40%, or 45%.

### 5.2.1.2 Aggregating agents

The aggregating agent can be an aggregating agent known in the art, such as those described in U.S. Pat. No. 5,482,829 and U.S. Patent Application Publication No. 2004/0142463, each incorporated herein by reference. Exemplary aggregating agents include dextran, hydroxyethyl starch (HES), gelatin, pentastarch, ficoll, gum ararbic, polyvinylpyrrolidone, Ficoll™-Hypaque, Histopaque®, 5-(N-2,3-dihydroxypropylacetamido)-2,4,6-tri-iodo-N,N′-bis(2,3 dihydroxypropyl) isophthalamide (Nycodenz®), Polymorphprep™, nucleic acids, proteins, and other natural or synthetic polymers. In some embodiments, the aggregating agent has a molecular weight of at least 40 kDa, e.g., between about 40 kDa and 2000 kDa, between 40, 50 or 60 kDa as the lower limit and 500 kDa as the upper limit or between 40, 50 or 60 kDa as the lower limit and 150 or 200 kDa as the upper limit, such as 70 kDa. In an embodiment, the aggregating agent is dextran, e.g., dextran having a molecular weight in a range as described in the preceding sentence.

The aggregating agent will generally, but not necessarily, be in an aqueous solution when combined with a sample comprising nucleated cells and non-nucleated red blood-cells. Suitable aqueous solutions include those identified in Section 5.2.1.1. In a preferred embodiment, the aggregating agent is dextran dissolved in RMPI media. In some embodiments, the same aqueous solution is used to prepare the sample comprising the nucleated cells and non-nucleated blood cells and to prepare the solution comprising the aggregating agent. By way of example, a sample comprising nucleated cells and non-nucleated red blood cells can be prepared by diluting an amount of blood with RPMI media, and a solution comprising the aggregating agent dextran can be prepared by dissolving an amount of dextran in RPMI media. The mixture to be separated can then be formed by combining the sample with an amount of the dextran solution.

The concentration of the aggregating agent in the mixture can affect rouleaux formation and their sedimentation rates. Suitable concentrations of aggregation agents are described in the art, for example, in U.S. Pat. No. 4,111,199, incorporated herein by reference, and can also be determined empirically. In some embodiments, the amount of aggregating agent in the mixture is 0.1-20%, 0.1-1%, 1-10%, 1-5%, 1%, 2%, 3%, 4%, or 5% (w/v). In a preferred embodiment, the mixture comprises 1% dextran (w/v).

### 5.2.1.3 Nucleated Cell Enriched Fractions

The nucleated cell enriched fractions obtained by the bulk separation processes described herein can comprise rare cell types such as stem cells, circulating cancer cells, or, in maternal blood, fetal nucleated cells (including fetal stem cells). The nucleated cell enriched fraction is depleted of most non-nucleated red blood cells. In some embodiments, the nucleated cell enriched fraction contains no more than 15%, no more than 10%, no more than 9%, no more than 8%, no more than 7%, no more than 6%, no more than 5%, no more than 4%, no more than 3%, no more than 2%, or no more than 1% of the non-nucleated red blood cells in the mixture used to make the nucleated cell enriched fraction. The nucleated cell enriched fraction comprises most of the nucleated cells in the mixture. In some embodiments, the nucleated cell enriched fraction contains at least 80%, at least 85%, at least 90%, at least 95%, at least 96%, at least 97%, at least 98%, at least 99%, or greater than 99% of the nucleated cells in the mixture. In some embodiments, the viability of the nucleated cells in the nucleated cell enriched fraction is greater than 90%, greater than 95%, greater than 96%, greater than 97%, greater than 98%, or greater than 99%.

A nucleated cell enriched fraction obtained by a process described herein can be further depleted of non-nucleated red blood cells by subjecting the nucleated cell enriched fraction to one, two, three, four, or more separations according to a process described in Section 5.2.1 to obtain a nucleated cell enriched fraction which is more highly enriched for nucleated cells and depleted of non-nucleated red blood cells. When repeating the separation step, the nucleated cell enriched fraction obtained from the first separation can be used to form the mixture for the second separation.

### 5.2.1.4 Devices for Separating Nucleated Cells from Non-Nucleated Red Blood Cells

The processes for bulk removal of non-nucleated red blood cells described herein can be performed using a separation device that comprises a lumen in which a mixture comprising nucleated cells and non-nucleated red blood cells can be separated. In a preferred embodiment, the lumen comprises a cylindrical section, although non-cylindrical sections of other geometries are also envisioned, e.g., a polyhedral section formed by connected polygons. In some embodiments, the cylindrical or non-cylindrical section is connected at its bottom end to a funnel-shaped section, e.g., a conical-shaped section, and/or connected at its top end to an inverted funnel-shaped section. The separation device can have one or more inlet/outlet ports that allow for the introduction and/or removal of liquid from the lumen, preferably located at the top and bottom of the lumen. When inlet/outlet ports are present, flow deflectors can be positioned within the lumen to deflect fluid introduced through an inlet/outlet port to prevent mixing of fluids within the lumen.

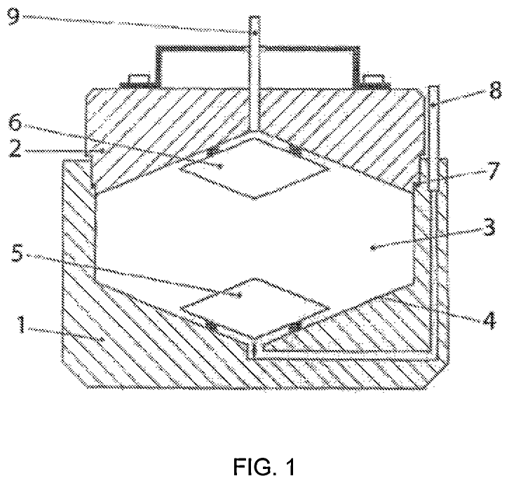

An exemplary separation device is shown in FIG. 1. The separation device shown in FIG. 1 comprises two cylindrical parts (1, 2) made, e.g., of transparent polycarbonate. Provided therein is a cylindrical chamber (3) whose bottom is conically shaped internally (4). Above the cylindrical chamber a conical flow-deflecting device (5) is situated. A cylindrical cover (2) whose internal surface is also conically shaped and provided with a conical flow-deflecting device (6) is also provided in this embodiment. The cover and the bottom part of the chamber can be attached to each other via screws and can be sealed via an O-ring (7). The flow deflecting device(s) is/are preferably arranged and fitted in such a way that a liquid flowing in or out (at the top and bottom) must flow through the narrow gap between the flow-deflecting device and the conical chamber wall. The initially high flow velocity is thus reduced, thus allowing the chamber to, e.g., be filled without flow disturbance. The inlet/outlet ports are preferably tube connections at the center of the chamber (8, 9).

An appropriate separation device can be sized based upon the volume of the mixture to be separated and the desired height of the mixture in the lumen. In some embodiments, the separation device is sized so that the volume of a mixture to be separated in the lumen has a height of 4 cm, less than 4 cm, less than 3.5 cm, less than 3 cm, less than 2 cm, less than 1.5 cm, or less than 1 cm. In some embodiments, the separation device is sized so that the average height of the mixture in the lumen is 1 cm, 1.5 cm, 2 cm, 2.5 cm, 3 cm, 3.5 cm, or 4 cm. In other embodiments, the separation device is sized so that the average height of the mixture in the lumen is 1-4 cm, 1-3 cm, or 1-2 cm. Preferably, the separation device is sized so that the average height of the mixture in the lumen is 1.5-2 cm.

For separation of a mixture in a cylindrical section of a lumen, the diameter of an appropriately sized lumen can calculated by the following formula:

$$= 2 \sqrt{\frac{V}{π h}} ,$$

where V is the volume of the mixture and h is the desired height of the mixture. By way of example, if using a separation device as shown in FIG. 1 to separate a 50 mL mixture with a desired height of no more than 2 cm, the diameter of the lumen should be at least about 5.6 cm.

For mixtures between 25 and 80 mL, cylinder diameters between 5 and 10 can be suitable. For mixtures between 20 and 60 mL, in particular between 45 and 55 mL, diameters of more than 5 cm, such as between 6 cm and 12 cm, between 7 cm and 9 cm, or 8 cm, can be particularly suitable. For larger volume mixtures, e.g., between 80 mL and 250 mL, cylinder diameters between 10 and 20 cm can be suitable. In some embodiments, separation devices of the disclosure comprise a lumen having a cylindrical section with a diameter of 1 to 20 cm, 3 to 8 cm, 4 to 9 cm, 5 to 20 cm, 5 to 10 cm, 6 to 12 cm, 7 to 14 cm, 8 to 12 cm, 8 to 16 cm, 10 to 15 cm, 10 to 20 cm, and in specific embodiment, the diameter is 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm.

When using a separation device as described herein, a heavy liquid can be added to the bottom of the lumen of the separation device prior to separation of the mixture comprising nucleated cells, non-nucleated red blood cells, and an aggregating agent. In some embodiments, the heavy liquid is a water immiscible liquid. The heavy liquid can have a density of at least 1.05 g/mL, at least 1.1 g/mL, least 1.2 g/mL, at least 1.5 g/mL, 1.75 g/mL or at least 2 g/mL, and can range up to 2.5 g/mL, 3 g/mL or even greater. In particular embodiments, the density ranges between any pair of the foregoing values, e.g., 1.05 g/mL to 1.1 g/mL, 1.05 g/mL to 1.2 g/mL, 1.05 g/mL to 1.5 g/mL, 1.5 g/mL to 2.5 g/mL, 1.2 g/mL to 2 g/mL, and so on and so forth. Suitable heavy liquids include heptacosafluorotributylamine (e.g., Fluorinert™ FC-43, 3M) and Ficoll solutions (e.g., Ficoll solutions having a density of 1.077 g/mL or 1.085 g/mL). The heavy liquid can be introduced to the lumen through an inlet/outlet port, if present. If the lumen has a lower funnel-shaped section, the amount of heavy liquid preferably fills at least the lower funnel-shaped section, and more preferably fills the entire lumen if an inlet/outlet port is present in the lower funnel-shaped section. If the heavy liquid does not fill then entire lower funnel-shaped section, the mixture, when introduced to the lumen, will have different heights at the periphery than in the center and the average height can need to be calculated. Subsequent to introducing the heavy liquid, the mixture is introduced to the lumen on top of the heavy liquid. If the lumen was filled completely with heavy liquid, heavy liquid is allowed to drain from the inlet/outlet port in the lower funnel shaped section as the mixture is introduced to the lumen. The mixture is then allowed to separate into a nucleated cell enriched fraction and a non-nucleated red blood cell enriched fraction in batch, under local gravity.

Following separation, the nucleated cell enriched fraction and/or the non-nucleated red blood cell enriched fraction can be collected from the separation device. If the separation device has an inlet/outlet port at the top of the lumen and an inlet/outlet port at the bottom of the lumen, the nucleated cell enriched fraction can be collected from the top inlet/outlet port by introducing additional heavy liquid through the bottom inlet/outlet port, thereby allowing the nucleated cell enriched fraction to be collected without significantly disturbing the interface between the nucleated cell enriched fraction and the non-nucleated red blood cell enriched fraction. Flow deflectors, when present, help to prevent mixing at the interface between the nucleated cell enriched fraction and the non-nucleated cell enriched fraction.

### 5.2.2 Target Nucleated Cell Enrichment

Nucleated cell enriched fractions, e.g., nucleated cell enriched fractions obtained by performing one or more separations according to a process described in Section 5.2.1 on a sample of blood or a sample of processed blood, can be further enriched for one or more target nucleated cell types, such as MSCs, nucleated red blood cells (NRBCs)), and CD34+ stem cells. When nucleated cell enriched fraction is prepared from maternal blood or umbilical cord blood, preferred target nucleated cell types comprise one or more fetal cell types such as fetal MSCs, fetal NRBCs, and fetal CD34+ stem cells. The further enrichment can comprise at least one (e.g., one, two, or all three) of negative selection for the target nucleated cells, positive selection for the target nucleated cells, and density gradient centrifugation. For example, the enrichment steps can be selected from the following combinations of negative selection, positive selection, and density gradient centrifugation:

(1) negative selection for the target nucleated cells followed by positive selection for the target nucleated cells;
(2) negative selection for the target nucleated cells followed by density gradient centrifugation;
(3) positive selection for the target nucleated cells followed by negative selection for the target nucleated cells;
(4) positive selection for the target nucleated cells followed by density gradient centrifugation;
(5) density gradient centrifugation followed by negative selection for the target nucleated cells;
(6) density gradient centrifugation followed by positive selection for the target nucleated cells;
(7) negative selection for the target nucleated cells followed by positive selection for the target nucleated cells followed by density gradient centrifugation;
(8) negative selection for the target nucleated cells followed by density gradient centrifugation followed by positive selection for the target nucleated cells;
(9) positive selection for the target nucleated cells followed by density gradient centrifugation followed by negative selection for the target nucleated cells;
(10) positive selection for the target nucleated cells followed by negative selection for the target nucleated cells followed by density gradient centrifugation;
(11) density gradient centrifugation followed by negative selection for the target nucleated cells followed by positive selection for the target nucleated cells; and
(12) density gradient centrifugation followed by positive selection for the target nucleated cells followed by negative selection for the target nucleated cells.

More than one negative selection step and/or more than one positive selection step (e.g., for different cell surface markers expressed by the target cell type(s)) can also be used to enrich for the target nucleated cells. Each negative selection step, positive selection step, and density gradient centrifugation step can, independently, be optionally preceded by or followed by one or more wash steps. A wash step can, for example, comprise combining the nucleated cell enriched fraction obtained from a positive selection step, a negative selection step, or a density gradient centrifugation step with a buffer or culture medium followed by centrifugation to pellet the nucleated cells. The cell pellet can then be resuspended in a suitable buffer or culture medium for further use or processing.

### 5.2.2.1 Negative Selection

Typically, the negative selection step(s) of the processes of the disclosure utilize one or more reagents that do not recognize target cells. The negative selection reagent can be any reagent that can be used to separate cells other than the target cells in a blood derived nucleated cell enriched fraction from the target cells.

In certain aspects, the reagent is a negative immunoselective antibody. Accordingly, the negative immunoselection can comprise the steps of: (a) contacting a blood derived nucleated cell enriched fraction with a negative immunoselective antibody in a fluid medium, wherein the negative immunoselective antibody selectively binds other cells in the biological sample relative to the target cells; and (b) selecting cells not bound to said negative immunoselective antibody. In some embodiments, the negative selection, if carried out, can be performed before, after, or concurrently with positive immunoselection, and can be performed before or after density gradient centrifugation.

When the blood derived nucleated cell enriched fraction is derived from maternal blood, the reagent is preferably an antibody that binds an antigen present on the cell surface of maternal cells, i.e. mature cells, but not present on the cell surface of fetal target cells.

When the target cells comprise MSCs (e.g., fetal MSCs) the one or more antibodies against cell surface markers selected not expressed by MSCs can be used. Exemplary cell surface markers include CD2, CD3, CD10, CD11b, CD14, CD15, CD16, CD19, CD31, CD34, CD35, CD38, CD44, CD45, CD49, CD49d, CD56, CD61, CD62(E), CD66b, CD68, CD79alpha, CD104, CD106, CD117, HLA-DR, and glycophorin A. In preferred embodiments, the negative immunoselection for MSCs utilizes one or more antibodies against one, two, three, four, five, or six of CD3, CD14, CD19, CD38, CD66b, and glycophorin A. An exemplary kit that can be used to further enrich a nucleated cell enriched fraction for MSCs is the RosetteSep™ Human Mesenchymal Stem Cell Enrichment Cocktail (Stemcell Technologies), which includes tetrameric antibody complexes recognizing CD3, CD14, CD19, CD38, CD66b, and glycophorin A.

When the target cells comprise fNRBCs, one or more negative immunoselective antibodies can be used, preferably against one or more haematopoietic cell surface markers. Exemplary cell surface markers include:(a) a T-lymphocyte cell surface marker such as CD3, CD4 or CD8; (b) a B-lymphocyte cell surface marker such as CD19, CD20, or CD32; (c) a pan lymphocyte marker such as CD45; (d) an NK cell surface marker such as CD56; (e) a dendritic cell surface marker such as CD11c or CD23; (f) a macrophage or monocyte cell surface marker such as CD14 or CD33; and (g) the “I” antigen. In particular embodiments, at least two, three, four, five, or six negative immunoselective antibodies are used.

### 5.2.2.2 Positive Selection

A positive selection reagent of the disclosure can be any reagent that can be used to distinguish target cells (e.g., fetal MSCs, NRBCs, and/or CD34+ stem cells) in blood derived nucleated cell enriched fraction from at least one other type of cell in the sample.

A preferred approach for target cell enrichment is the use of positive immunoselection methods carried out in a fluid medium. Typically, the positive immunoselection methods utilize a positive immunoselective antibody. In certain aspects, a plurality of positive immunoselective antibodies are used in a positive immunoselection procedure. Accordingly, positive immunoselection can comprise the steps of: (a) contacting blood derived nucleated cell enriched fraction with a positive immunoselective antibody in a fluid medium, wherein the positive immunoselective antibody selectively binds to the target cells relative to one or more other cell types in the blood derived nucleated cell enriched fraction; and (b) selecting cells bound to said positive immunoselective antibody.

Positive selection markers for MSCs include CD73, CD90, CD105, CD166, CD200, CD271, STRO-1, and “i” antigen. In particular, MSCs isolated from umbilical cord blood are “i”—antigen positive (Hirvonen et al., 2102, Stem Cell Dev., 21(3):455-64). Monoclonal antibodies against the “i” antigen are known in the art (Hirvonen et al., 2013 Biores open Access, 2(5):336-45). Lectins can also be used for positive selection of “i” positive cells. In preferred embodiments, anti-CD73 and/or anti-i antibodies are used to positively select for MSCs.

Positive selection markers for fNRBCs include glycophorin A (also known as CD235a), “i” antigen, CD36, CD71, and nuclear markers. Where the downstream analysis permits cell fixation (e.g., FISH), fetal hemoglobin can be a positive selection marker. Cells expressing the markers glycophorin A, “i” antigen, CD36, CD71 and fetal hemoglobin can be selected (e.g., sorted or enriched for) using antibodies against the markers. In contrast to maternal erythrocytes, fNRBCs are nucleated and can be selected using nuclear dyes, such as Hoechst 33342, LDS751, TO-PRO, DC-Ruby, and DAPI.

In some embodiments, fNRBCs are selected for using the monoclonal antibody 4B9. The hybridoma producing the antibody 4B9 is deposited at the Deutsche Sammlung von Mikroorganismen and Zelkulturen GmbH under accession number DSM ACC 2666 fNRBCs (see U.S. Pat. Nos. 7,858,757 B2 and 8,563,312 B2 of Hollmann et al.). In other embodiments, fNRBCs are selected for using an antibody that competes with 4B9 for binding to the surface of fNRBCs. By way of example, monoclonal antibody 4B8 competes with 4B9 for binding to fNRBCs (see U.S. Pat. Nos. 7,858,757 B2 and 8,563,312 B2 of Hollmann et al.).

In preferred embodiments, an anti-i antibody and/or the 4B9 antibody are used to positively select for fNRBCs.

### 5.2.2.3 Immunoselection Techniques

Immunoselection steps can utilize immunodensity separation, e.g., using bispecific tetrameric antibody complexes (TACs) that crosslink non-target cells to a particle (e.g., another non-target cell), flow cytometry, or magnetic separation, e.g., using antibody-coated magnetic beads. Advantageously, immunodensity separation using a cocktail comprising bispecific TACs that crosslink non-target nucleated cells to non-nucleated red blood cells present in the nucleated cell enriched fraction can be used to remove both unwanted nucleated cells and non-nucleated red blood cells from the nucleated cell enriched fraction. An exemplary TAC cocktail that can be used to negatively select for MSCs is the RosetteSep™ Human Mesenchymal Stem Cell Enrichment Cocktail (Stemcell Technologies). Flow cytometric techniques can provide accurate separation via the use of, e.g., fluorescence activated cell sorters, which can have varying degrees of sophistication, such as multiple color channels, low angle and obtuse light scattering detecting channels, impedance channels, etc.

In some embodiments, both negative selection using immunodensity separation and positive selection using FACS are used in the processes of the disclosure to isolate a population of target nucleated cells.

Antibodies can be conjugated with labels, e.g., magnetic beads and fluorochromes, to allow for ease of separation of the target cells from other cells types. Fluorochromes can be used with a fluorescence activated cell sorter. Multi-color analyses can be employed with the FACS or in a combination of immunomagnetic separation and flow cytometry. Multi-color analysis is of interest for the separation of cells based on multiple surface antigens. Fluorochromes which find use in a multi-color analysis include phycobiliproteins, e.g., phycoerythrin and allophycocyanins; fluorescein and Texas red. A negative designation indicates that the level of staining is at or below the brightness of an isotype matched negative control. A dim designation indicates that the level of staining may be near the level of a negative stain, but may also be brighter than an isotype matched control. A positive immunoselective antibody of the disclosure preferably gives rise to a “bright” designation with respect to target cells and a “negative” or “dim” designation with respect to one or more (and in some embodiments all) other cell types that can be present in a nucleated cell enriched fraction in which the target cells are present. A negative immunoselective antibody of the disclosure preferably gives rise to a “negative” or “dim” designation with respect to target cells and a “bright” designation with respect to one or more other cell types that can be present in a nucleated cell enriched fraction in which the target cells are present.

In one embodiment, an immunoselective antibody is part of a monospecific or bispecific TAC. For example, a mono-specific TAC comprising two antibodies targeting non-nucleated red blood cells can be used to cross-link two non-nucleated red blood cells to each other. A bispecific TAC comprising an antibody targeting a non-nucleated red blood cell and an antibody targeting a non-target nucleated cell can be used to cross-link a non-nucleated red blood cell to a non-target nucleated cell. In some embodiments, a cocktail comprising bispecific TACs targeting different non-target cell types and a mono-specific TAC targeting non-nucleated blood cells is used to negatively select for the target cell type(s). Complexes of cross-linked non-nucleated red blood cells and non-target nucleated cells can be separated from the target nucleated cells by density gradient centrifugation.

In another embodiment, an immunoselective antibody is directly or indirectly conjugated to a magnetic reagent, such as a superparamagnetic microparticle (microparticle). Direct conjugation to a magnetic particle is achieved by use of various chemical linking groups, as known in the art. The antibody can be coupled to the microparticles through side chain amino or sulfhydryl groups and heterofunctional cross-linking reagents. A large number of heterofunctional compounds are available for linking to entities. A preferred linking group is 3-(2-pyridyidithio)propionic acid N-hydroxysuccinimide ester (SPDP) or 4-(N-maleimidomethyl)-cyclohexane-1-carboxylic acid N-hydroxysuccinimide ester (SMCC) with a reactive sulfhydryl group on the antibody and a reactive amino group on the magnetic particle.

Alternatively, an immunoselective antibody is indirectly coupled to the magnetic particles. The antibody is directly conjugated to a hapten, and hapten-specific, second stage antibodies are conjugated to the particles. Suitable haptens include digoxin, digoxigenin, FITC, dinitrophenyl, nitrophenyl, avidin, biotin, etc. Methods for conjugation of the hapten to a protein are known in the art, and kits for such conjugations are commercially available.

To practice the positive immunoselection method, a positive immunoselective antibody is added to a nucleated cell enriched fraction. The amount of antibody necessary to bind target cells can be empirically determined by performing a test separation and analysis. The cells and antibody are incubated for a period of time sufficient for complexes to form, usually at least about 5 minutes, more usually at least about 10 minutes, and usually not more than one hour, more usually not more than about 30 minutes.

The nucleated cell enriched fraction may additionally be incubated with additional positive and/or negative immunoselective antibodies as described herein. The labeled cells are separated in accordance with the specific antibody preparation. Fluorochrome-labeled antibodies are useful for FACS separation, magnetic particles for immunomagnetic selection, particularly high gradient magnetic selection (HGMS), etc. Exemplary magnetic separation devices are described in WO 90/07380, PCT/US96/00953, and EP 438,520.

The positive immunoselection and/or negative immunoselection can be performed using other automated methods, such as ultrafiltration or microfluidic separation.

### 5.2.2.4 Density Gradient Centrifugation

Density gradient separation is a technique that allows the separation of cells depending on their size, shape and density. A discontinuous density gradient is created in a centrifuge tube by layering solutions of varying densities with the dense end at the bottom of the tube. Cells are usually separated on a shallow gradient of sucrose or other inert carbohydrates even at relatively low centrifugation speeds.

Discontinuous density gradient centrifugation is commonly used to isolate peripheral blood mononuclear cells from granulocytes and erythrocytes. For example, in a so called Ficoll density separation, a sample, such as whole blood, is layered over FICOLL-PAQUE® and then centrifuged. The erythrocytes, granulocytes and a portion of the mononuclear cells settle to the cell pellet while the remaining mononuclear cells settle to the Ficoll plasma interface. Density gradient centrifugation can be performed, for example, using a device as described in U.S. Pat. No. 6,309,606, which is incorporated herein by reference in its entirety.

Density gradient centrifugation of a nucleated cell enriched fraction can be preceded by a step of incubating the nucleated cell enriched fraction in a medium having non-physiological conditions in order to alter the density of cells in the nucleated cell enriched fraction to allow for improved separation of target cells from non-target cells (see, Sitar et al., 2005, Experimental Cell Research 302:153-161, incorporated herein by reference in its entirety). The use of non-physiological conditions is particularly useful for enriching a nucleated cell enriched fraction derived from maternal blood for fetal cells (such as fNRBCs, fMSCs, and CD34+ fetal stem cells). Non-physiological conditions can comprise a non-physiological pH and/or non-physiological osmolarity. In some embodiments, a medium having non-physiological conditions can have a pH in the range of 6.1 to 6.9, 6.2 to 6.8, or 6.3 to 6.7 (e.g., about 6.1, about 6.2, about 6.3, about 6.4, about 6.5, about 6.6., about 6.7, about 6.8, or about 6.9). In some embodiments, a medium having non-physiological conditions can have an osmolarity in the range of 270 to 370 mOsm/l, 270 mOsm/l to 280 mOsm/l, 280 mOsm/l to 290 mOsm/l, 300 mOsm/l to 310 mOsm/l, 310 mOsm/l to 320 mOsm/l, 320 mOsm/l to 330 mOsm/l, 330 mOsm/l to 340 mOsm/l, 340 mOsm/l to 350 mOsm/l, 350 mOsm/l to 360 mOsm/l, or 360 mOsm/l to 370 mOsm/l.

Non-physiological conditions can be created, for example, by combining a nucleated cell enriched fraction with a medium comprising citric acid, sodium citrate and dextrose (ACD), which has optionally had its osmolarity adjusted with NaCl. In preferred embodiments, the ACD medium is characterized by one, two, three, four, five, six, or seven of the following: a pH of 6.4 to 6.6, an osmolarity of 300 to 330 mOsm, a sodium concentration of 150 to 170 mmol/l, a potassium concentration of 4.5 to 5.5 mmol/l, a chloride concentration of 100 to 115 mmol/l, a calcium concentration of 1 to 2.5 mmol/l, and a dextrose concentration of 400 to 500 mg/dl.

### 5.3 Populations of Target Nucleated Cells

The disclosure provides populations of target nucleated cells obtained by the processes of the disclosure. A population of target nucleated cells can be cultured to maintain or expand the population of target cells. Populations of target nucleated cells can be cultured using standard cell culture media and techniques. For example, populations comprising MSCs can be cultured using the MesenCult™-ACF Culture Kit (Stemcell Technologies) according to the manufacturer's instructions. In some embodiments, selective media is used to maintain or expand the population of target cells while preventing the expansion of non-target cells in the population. For example, a population containing NRBCs, MSCs and CD34+ cells can be cultured to selectively expand a desired target cell type by adding growth factors to the culture medium that favor the growth of the target cell type. For example, NRBCs can be expanded by culturing the population in a medium comprising erythropoietin and Fe²⁺. Alternatively, MSC or CD34+ cells can be expanded by culturing the population in the presence of growth factors specific for MSC or CD34+ cells, respectively.

In some embodiments, platelet lysate is added to the culture medium during culturing to maintain or expand the population of target cells.

In some embodiments, a population of target nucleated cells comprising fetal cells is cultured in a MSC medium comprising human stem cell factor (SCF), interleukin 6 (IL-6), FMS-like tyrosine kinase 3 (Flt-3) ligand and megakaryocyte growth and development factor (MGDF) to promote the expansion of MSCs.

In other embodiments, a population of target nucleated cells comprising fetal cells is cultured in a medium comprising erythropoietin and Fe²⁺ to promote the expansion of fNRBCs. In other embodiments, a population of target nucleated cells comprising fNRBCs is cultured in a medium comprising erythropoietin and heme to promote expansion of fNRBCs.

The target nucleated cells in populations of target nucleated cells preferably comprise a majority of the cells in the population, e.g., 60% to 100%, 60% to 70%, 70% to 80%, 80% to 90%, or 90% to 100%. In some embodiments, at least 70%, at least 80%, at least 90%, or at least 95% of the cells in the population are target cells.

Following culturing (e.g., from 3 to 9 days), single cells or groups of cells in the population can be analyzed to confirm their identity as target cells, and/or used for diagnosis or therapy as described in Section 5.4. In some embodiments, cells or groups or cells are cultured from 3 to 14 days before analysis and/or use in diagnosis or therapy, e.g., 3 days, 4 days, 5 days, 6 days, 7 days, 8 days, 9 days, 10 days, 11 days, 12 days, 13 days, 14 days, or for any range of the foregoing number of days, such as 3 to 7 days, 7 to 10 days, or 10 to 14 days.

FISH can be used to validate a cell or group of cells isolated by the methods described herein as a fetal cell(s). Genetic fingerprinting methods that involve, for example, generating a genetic profile using Short Tandem Repeat (STR) analysis, Restriction Fragment Length Polymorphism (RFLP) analysis or Single Nucleotide Polymorphism (SNP) analysis can also be used to validate a fetal cell or cells. By comparing the profile generated from the isolated cell(s) to a profile generated from maternal and optionally, paternal cells, the identity of the isolated cell(s) as a fetal cell(s) can be verified. Suitable kits for generating genetic profiles are commercially available. For example, the PowerPlex® Fusion STR kit from Promega and the Genome-Wide Human SNP Array 6.0 from Affymetrix can be used to generate STR and SNP profiles, respectively, which can be used to validate the identity of fetal cells. In some embodiments, whole genome amplification (WGA) is used to increase the amount of genetic material available for analysis.

### 5.4 Uses

### 5.4.1 Diagnostic Uses

Populations of target nucleated cells of the disclosure and individual cells isolated from a population of target nucleated cells can be used for genetic testing. In particular, fetal cells (e.g., fetal MSCs, fNRBCs, and fetal CD34+ stem cells) isolated from a sample of maternal blood, or progeny thereof, can be used for prenatal genetic testing to identify a fetal abnormality. In some embodiments, the fetal cell(s) used for prenatal genetic testing comprise one, two, or all three of fetal MSCs, fNRBCs and fetal CD34+ stem cells isolated from maternal blood, or progeny thereof.

Examples of abnormalities that can be tested for include trisomy 13, trisomy 18, trisomy 21, Down syndrome, neuropathy with liability to pressure palsies, neurofibromatosis, Alagille syndrome, achondroplasia, Huntington's disease, alpha-mannosidosis, beta-mannosidosis, metachromatic leucodystrophy, von Recklinghausen's disease, tuberous sclerosis complex, myotonic dystrophy, cystic fibrosis, sickle cell disease, Tay-Sachs disease, beta-thalassemia, mucopolysaccharidoses, phenylketonuria, citrullinuria, galactosemia, galactokinase and galactose 4-epimerase deficiency, adenine phosphoribosyl, transferase deficiency, methylmalonic acidurias, proprionic acidemia, Farber's disease, fucosidosis, gangliosidoses, gaucher's disease, I cell disease, mucolipidosis III, Niemann-Pick disease, sialidosis, Wolman's disease, Zellweger syndrome, cystinosis, factor X deficiency, ataxia telangiectasia, Bloom's syndrome, Robert's syndrome, xeroderma pigmentosum, fragile (X) syndrome, sex chromosome aneuploidy, Klinefelter's Syndrome, Turner's syndrome, XXX syndrome, steroid sulfatase deficiency, microphthalmia with linear skin defects, Pelizaeus-Merzbacher disease, testis-determining factor on Y, ornithine carbamoyl transferase deficiency, glucose 6-phosphate dehydrogenase deficiency, Lesch-Nyhan syndrome, Anderson-Fabry disease, hemophilia A, hemophilia B, Duchenne type muscular dystrophy, Becker type muscular dystrophy, dup(17)(p11.2p11.2) syndrome, 16p11.2 deletion, 16p11.2 duplication, Mitochondrial defect, dup(22)(q11.2q11.2) syndrome, Cat eye syndrome, Cri-du-chat syndrome, Wolf-Hirschhorn syndrome, Williams-Beuren syndrome, Charcot-Marie-Tooth disease, chromosome rearrangements, chromosome deletions, Smith-Magenis syndrome, Velocardiofacial syndrome, DiGeorge syndrome, 1p36 deletion, Prader-Willi syndrome, Azospermia (factor a), Azospermia (factor b), Azospermia (factor c), spina bifida, anencephaly, neural tube defect, microcephaly, hydrocephaly, renal agenesis, Kallmann syndrome, Adrenal hypoplasia, Angelman syndrome, cystic kidney, cystic hygroma, fetal hydrops, exomphalos and gastroschisis, diaphragmatic hernia, duodenal atresia, skeletal dysplasia, cleft lip, cleft palate, argininosuccinicaciduria, Krabbe's disease, homocystinuria, maple syrup urine disease, 3-methylcrotonyl coenzyme A, carboxylase deficiency, Glycogenoses, adrenal hyperplasia, hypophosphatasia, placental steroid sulphatase deficiency, severe combined immunodeficiency syndrome, T-cell immunodeficiency, Ehlers-Danlos syndrome, osteogenesis imperfect, adult polycystic kidney disease, Fanconi's anemia, epidermolysis bullosa syndromes, hypohidrotic ectodermal dysplasia, congenital nephrosis (Finnish type) and multiple endocrine neoplasia.

The diagnostic assay can be a nucleic acid (e.g., DNA or RNA) assay, a protein (e.g., antibody-based) assay, or a histology assay, or a combination thereof. Examples of DNA assays include FISH, PCR and DNA sequencing assays. Examples of RNA assays include RT-PCR assay and FISH assays. To facilitate access to the nucleic acid, the target cells can be lysed or permeabilized prior to carrying out the diagnostic test. The DNA, RNA and protein assays can be performed on a microarray. Illustrative methods are described below.

In some embodiments, genomic DNA from single cells or groups of two to four or more cells can be amplified by whole genome amplification (WGA) to provide sufficient nucleic acid for analysis. Groups of cells containing 5 or more fetal cells can be analyzed without the use of whole genome amplification (WGA). WGA refers to amplification of the entire genome of a cell or group of cells of an individual. For example, a whole genome can be amplified using the genetic material of a single cell (i.e., single cell whole genome amplification (SCWGA)). In other embodiments, a subset of the genome can be amplified prior to analyzing the DNA.

Chromosomal abnormalities, single gene abnormalities, allelic variants and single nucleotide polymorphisms (SNPs) are detectable using the chromosomes or nucleic acid from lysed fetal NRBCs produced by the methods of the present disclosure by any of a variety of methods, including fluorescence in situ hybridization (FISH), polymerase chain reaction (PCR), multiple annealing and looping based amplification cycles (MALBAC), restriction fragment length polymorphism (RFLP) analysis and DNA sequencing. The PCR technique can be a simple PCR amplification technique or a quantitative PCR, a real-time PCR or a reverse transcriptase PCR technique. Other useful genetic analysis techniques include array comparative genomic hybridization (CGH) and analysis in a DNA microarray. For instance, the fetal cells can be analyzed in a prenatal chromosomal microarray.

A haplotype is a combination of alleles that occur together and at adjacent locations on a chromosome. A haplotype may be found on a single locus or on several loci. Haplotypes may occur throughout an entire chromosome. Haplotypes may include any number of recombination events. A haplotype may also refer to a set of associated single nucleotide polymorphisms.

A single nucleotide polymorphism (SNP) occurs where there is a variation from a normal (e.g., wild type) nucleotide sequence in a single nucleotide (e.g., A, T, C or G). For example, a single nucleotide polymorphism may result in an allelic variant. A given allele may be defined by a single nucleotide polymorphism or by multiple nucleotide changes.

Restriction Fragment Length Polymorphisms (RFLPs) are differences in homologous sequences of DNA. They may be detected by differences in fragment lengths found after digestion of DNA using a particular restriction endonuclease or combination of restriction endonucleases. RFLP may be determined by gel electrophoresis or southern blots.

Fluorescence in situ hybridization (FISH) is performed by binding fluorescent probes to a portion of a fixed nucleic acid sequence complement to that of the fluorescent probe. FISH can be used to fluorescently tag a target nucleic acid sequence in RNA or DNA at the specific position where a nucleic acid sequence occurs within a larger nucleic acid sequence. For example, FISH may be used to tag a target sequence on a chromosome. The fluorescent probe may be viewed using fluorescence microscopy.

PCR is used to amplify one or more copies (i.e., amplicons) of a particular nucleic acid sequence by using two primers. PCR methods are readily available and are commonly used to diagnose hereditary diseases.

Quantitative PCR (qPCR) is based on a polymerase chain reaction (PCR) and is used to both amplify and simultaneously quantify the total number of copies or the relative number of copies of a nucleic acid sequence. One example of qPCR is Real-Time PCR. In Real-Time PCR, the number or relative number of nucleic acid copies resulting from PCR are detected in real time. The number or relative number of copies produced by qPCR may be detected and quantified using a signal generated by fluorescent dyes.

Reverse Transcription Polymerase Chain Reaction (RT-PCR) is a method which can be used to detect RNA molecules or to determine the expression levels of a specific RNA sequence (e.g., mRNA) by transcribing the RNA molecule(s) into DNA copies (cDNA) and amplifying the DNA. RT-PCR may be performed by a one-step or two-step process.

Array Comparative Genomic Hybridization (array CGH) is a microarray technique used to determine chromosome copy number variations that occur on a genome-wide scale. Array CGH compares a test genome with a normal (e.g., wild type) genome to detect even relatively small (e.g., 200 base pairs) structural variations. For example, array CGH may detect deletions, amplifications, breakpoints or aneuploidy. Array CGH may also be used to detect a predisposition for developing a cancer.

Multiple Annealing and Looping Based Amplification Cycles (MALBAC) is a whole genome amplification method. MALBAC can be used for single cell, whole genome amplification. MALBAC can be used to amplify a genome in a quasi-linear fashion and avoid preferential amplification of certain DNA sequences. In MALBAC, amplicons may have complementary ends, which form loops in the amplicon and therefore prevent exponential copying of the amplicon. Amplicon loops may prevent amplification bias. MALBAC can be applied to diagnosing fetal abnormalities using a single fetal cell, or may be used to identify a fetal predisposition for developing a cancer using a single fetal cell.

Next Generation Sequencing (NGS) is a group of high-throughput sequencing technologies that can be used for detecting a fetal abnormality. NGS (e.g., massively parallel sequencing) uses a cell sample as small as a single cell to sequence large stretches of nucleic acid sequences or an entire genome. For example, in NGS many relatively small nucleic acid sequences may be simultaneously sequenced from a genomic DNA (gDNA) sample from a library of small segments (i.e., reads). The reads can then be reassembled to identify a large nucleic acid sequence or a complete nucleic acid sequence of a chromosome. For instance, in NGS, as many as 500,000 sequencing operations may be run in parallel. NGS is a form of single cell, whole genome amplification (WGA). For instance, MALBAC may be used for NGS when followed by traditional PCR.

Massively Parallel Signature Sequencing (MPSS) is one example of an NGS. MPSS identifies mRNA transcripts from 17-20 base pair signature primer sequences. MPSS can be utilized to both identify and quantify mRNA transcripts in a sample (Brenner et al., 2000, Nature biotechnology 18(6):630-634).

Polony Sequencing is another example of NGS. Polony sequencing can be used to read millions of immobilized DNA sequences in parallel. Polony sequencing is a multiplex sequencing technique that has been found to be extremely accurate (low error rate) (Shendure et al., 2004. Nature Reviews Genetics 5(5):335-344, 2004; Shendure et al., 2008, Nature Biotech 26(10):1135-1145).

454 Pyrosequencing is another example of NGS. 454 pyrosequencing utilizes luciferase to detect individual nucleotides added to a nascent DNA. 454 pyrosequencing amplifies DNA contained in droplets of water in an oil solution. Each droplet of water contains one DNA template attached to a primer-coated bead (Vera et al., 2008, Molecular Ecology 17(7):1636-1647).

IIlumina Sequencing is another example of NGS. In Illumina Sequencing DNA molecules and primers are attached to a slide. The DNA molecules are amplified by a polymerase and DNA colonies (DNA clusters) are formed (Shendure et al., 2008, Nature Biotech 26(10):1135-1145; Meyer et al., 2010, Cold Spr Hbr Protocols 2010(6):pdb-prot 5448).

Sequencing by Oligonucleotide Ligation and Detection (SOLiD Sequencing) is another example of NGS. SOLiD sequencing is a method of sequencing by ligation. SOLiD sequencing randomly generates thousands of small sequence reads simultaneously and immobilizes the DNA fragments on a solid support for sequencing (Shendure et al., 2008, Nature Biotech 26(10):1135-1145; Meyer et al., 2009, New Biotechnology 25(4):195-203).

Ion Torrent Semiconductor Sequencing is another example of NGS. Ion Torrent Semiconductor Sequencing is a sequencing-by-synthesis method that detects hydrogen ions released during DNA polymerization. A deoxyribonucleotide triphosphate is introduced into a microwell containing a template DNA strand to be sequenced. When the dNTP is complementary to a leading template nucleotide, the dNTP is incorporated into the complementary DNA strand and a hydrogen ion is released (Quail et al., 2012, BMC Genomics 13(1):341).

DNA Nanoball Sequencing is another example of NGS. DNA Nanoball Sequencing can be used to determine an entire genomic sequence of an organism, such as, for instance, a newly discovered organism. Small fragments of genomic DNA are amplified using rolling circle replication to form DNA nanoballs. DNA sequences can then be ligated by using fluorescent probes as guides (Ansorge et al., 2009, New Biotechnology 25(4):195-203; Drmanac et al., 2010, Science 327(5961):78-81).

Heliscope Single Molecule Sequencing is another example of NGS. Heliscope Single Molecule Sequencing is a direct-sequencing approach that does not require ligation or PCR amplification. DNA is sheared, tailed with a poly-A tail and then hybridized to the surface of a flow cell with oligo(dT). Billions of molecules may be then sequenced in parallel (Pushkarev et al., 2009, Nature Biotechnology 27(9):847-850).

Single Molecule Real Time (SMRT) Sequencing is another example of NGS. SMRT sequencing is a sequencing-by-synthesis approach. DNA is synthesized in small well-like containers called zero-mode wave-guides (ZMWs). Unmodified polymerases attached to the bottom of the ZMWs are used to sequence the DNA along with fluorescently labeled nucleotides which flow freely in the solution. Fluorescent labels are detached from the nucleotides as the nucleotide is incorporated into the DNA strand (Flusberg et al., 2010, Nature methods 7(6):461-465).

Ultra-Deep Sequencing refers to the number of times that a nucleic acid sequence is determined from many template copies. Ultra-Deep Sequencing may be used to identify rare genetic mutations by amplifying a relatively small target nucleic acid sequence which may contain a rare mutation.

DNA Microarray can be used to measure the expression levels of multiple genes simultaneously. DNA Microarray can also be used to genotype multiple regions of a genome. For example, Prenatal Chromosomal Microarray (CMA)—can be used to detect copy-number variations, such as aneuploidies in a chromosome. Prenatal CMA may detect deletions or duplications of all or part of a chromosome.

In certain aspects, a single cell or a small group of cells can be subject to DNA fingerprinting, for example on a SNP microarray using the principles described by Treff et al., 2010, Fertility and Sterility 94(2):477-484, which is incorporated by reference herein in its entirety. The SNP microarrays to be used in these methods are preferably genome-wide SNP arrays. In various embodiments, the SNP fingerprint comprises at least 50,000, at least 100,000, at least 150,000, at least 200,000 or at least 250,000 SNPs. The SNP fingerprint can be generated from a single microarray or multiple microarrays. Using comparative DNA fingerprinting, a fetal cell can be distinguished from a maternal cell. In preferred embodiment, the determination of a match with the maternal cell (e.g., that the cell under examination is a maternal, rather than fetal, cell) is based on at least 1,000, more preferably at least 1,500 and yet more preferably at least 2,000 informative SNPs. The maternal fingerprint can be based on a historical maternal sample or a maternal sample run in parallel with the fetal cell. The DNA fingerprinting can be preceded by WGA of the fetal cell and optionally the maternal sample. The SNP fingerprint can also be used to fetal abnormalities or other characteristics. Microarrays can be adapted to include a combination of SNPs and markers of fetal characteristics and/or possible fetal cell abnormalities, such as those described above. In particular embodiments, the microarrays include at least 5, at least 10, at least 15, at least 20, at least 30 or at least 50 markers of possible fetal cell abnormalities and/or markers of fetal sex, such as Y chromosome markers.

### 5.4.2 Therapeutic Uses

The disclosure further provides methods for in utero stem cell therapy comprising delivering a population of fetal stem cells (e.g., MSCs and/or CD34+ stem cells) obtainable by a process of the disclosure to a fetus in utero. Populations of fetal stem cells may be useful for treating hematological diseases (e.g., alpha-thalassemia (O'Brien et al., 2005, Clinical Obstetrics and Gynecology 4:885-896)), metabolic diseases, immunological diseases (e.g., severe combined immunodeficiency), bone disorders (e.g., osteogenesis imperfecta (Chan et al., 2014, Frontiers in Pharmacology 5:223), neural tube defects (Li et al., 2012, J. Cell. Mol. Med. 16(7):1606-1617), and to correct a genetic defect (O'Brien et al., 2005, Clinical Obstetrics and Gynecology 4:885-896; Chan et al., 2005, Stem Cells 23:93-102).

The disclosure further provides methods for stem cell therapy in a juvenile or adult individual in need of stem cell therapy comprising delivering a population of stem cells (e.g., MSCs) obtainable by a process of the disclosure to the individual. Populations of stem cells may be useful for treating wounds, orthopedic injuries, cardiovascular diseases, autoimmune diseases, liver disease, neurological disorders, neuronal degeneration, graft versus host disease, metabolic diseases, renal infarction, myocardial infarction, supporting hematopeietic engraftment, and regeneration of bone and/or cartilage tissues (Farini et al., 2014, Stem Cells International, Article ID 306573; Kim et al., 2013, Korean J. Intern. Med. 28:387-402; Phinney et al., 2014, Brain Res. 0:92-107; Lange et al., 2007, J. Cell. Physiol. 213:18-26).

In some embodiments, the populations of stem cells used for stem cell therapy comprise allogeneic stem cells. In other embodiments, the population of stem cells used for stem cell therapy comprise autologous stem cells made recombinant by introduction of a gene of which the fetus, juvenile, or adult is in need (Chan et al., 2008 Hum. Reprod., 23(11):2427-2437).

### 5.5 Exemplary Protocols

### 5.5.1 Bulk RBC Removal

The following separation protocol can be used to obtain a nucleated cell enriched fraction from whole blood.
1. Dilute an amount of blood with an aqueous solution.
2. Centrifuge the diluted blood to form a cell pellet containing nucleated cells and non-nucleated red blood cells and remove some or all of the platelet rich plasma.
3. Resuspend the cell pellet in an aqueous solution.
4. Add a pre-made solution of dextran to the resuspended cells to form a mixture containing, e.g., dextran at a final concentration of 1% (w/v).
5. Add a volume of the mixture to a sedimentation separation device prefilled with heavy liquid to attain a mixture column height of 1.5-2 cm, while simultaneously draining a volume of heavy liquid from the device equal to the volume of the mixture.
6. Allow the mixture to separate into a nucleated cell enriched fraction and a non-nucleated cell enriched fraction.
7. Collect the nucleated cell enriched fraction from the sedimentation separation device.

### 5.5.2 Target Nucleated Cell Enrichment

The following enrichment protocol can be used to enrich a nucleated cell enriched fraction using negative selection and density gradient centrifugation, e.g., a nucleated cell enriched fraction obtained by the bulk RBC removal protocol of Section 5.5.1, for target nucleated cells.
1. Wash a nucleated cell enriched fraction by combining the nucleated cell enriched fraction with a physiological solution, e.g., RPMI, and then centrifuging the resulting mixture to pellet the cells.
2. Resuspend the cell pellet in an aqueous solution, e.g., platelet-free plasma, then centrifuge the resulting mixture to pellet the cells.
3. Resuspend the cell pellet from step 2 in an aqueous solution, e.g., platelet rich plasma separated during the RBC bulk removal process.
4. Combine the resuspended cells from step 3 with a negative selection reagent for under mild rotation to favor binding of the negative selection reagent to unwanted cells.
5. Subject the mixture of step 4 to density gradient centrifugation (e.g., with a density cut of 1.082).
6. Collect the cell fraction enriched in target nucleated cells.
7. Wash the fraction enriched in target nucleated cells in a manner similar to steps 1 and 2.

The above protocol can be used to obtain a fraction containing one, two, or all three of fNRBCs, fMSCs and CD34+ stem cells, depending on the choice of negative selection reagent.

### 5.5.3 Cell Culture

The following cell culture protocol can be used to culture mesenchymal stem cells, e.g., obtained by the target nucleated cell enrichment protocol of Section 5.5.2.
1. Resuspend a cell pellet enriched in mesenchymal stem cells in mesenchymal stem cell culture medium.
2. Transfer the cell suspension to a chamber slide or cell culture dish and incubate the slide or dish at 37° C.
3. Replace the culture medium every three days.

### 6. EXAMPLES

### 6.1 Example 1: Separation of Nucleated Cells from Maternal Blood

A separation device of the type shown in FIG. 1 having a lumen 8 cm in diameter was filled with Fluorinert™ FC-43. 25 mL of peripheral blood obtained from a pregnant female was combined with 25 mL of RPMI media comprising 2% dextran (w/v) to provide a mixture having a final dextran concentration of 1%. The mixture was introduced to the lumen of the separation device through the top inlet/outlet port on top of the FC-43 down to the cylindrical part of the separation device and allowed to separate under local gravity into a nucleated cell enriched fraction and a non-nucleated red blood cell enriched fraction for 9 minutes. The nucleated cell enriched fraction was then collected from the top inlet/outlet port by introducing additional FC-43 into the lumen through the bottom inlet/outlet port.

A second separation was performed using the nucleated cell enriched fraction to deplete the sample of remaining non-nucleated red blood cells. After the first separation, the nucleated cell enriched fraction was mixed with RPMI medium containing 1% dextran and introduced again into the lumen of the separation device (after removal of the red blood cells of the first separation) and allowed to sediment under local gravity, to obtain a nucleated cell enriched fraction almost entirely free of red blood cells.

The percent recovery of nucleated cells and non-nucleated red blood cells in the nucleated cell enriched fraction following the first and second separations is shown below in Table 1.

TABLE 1
Cell recovery from maternal blood subjected
to two cycles of separation

| | Percent recovery in the | Percent recovery in the | |
|---|---|---|---|
| | NC enriched fraction | NC enriched fraction | |
| | after one separation | after two separations | p value |
| RBCs | 4.7 ± 1.8 | 1.6 ± 1.2 | <0.005 |
| NCs | 93.6 ± 6.4 | 98.6 ± 5.8 | <0.005 |
| NC viability | 98.7 ± 1.6 | 99.1 ± 1.3 | <0.005 |

### 6.2 Example 2: Isolating a Population of Target Nucleated Cells

Samples of maternal blood from pregnant women at 11-14 weeks of normal gestation were processed as described in Example 1 to obtain nucleated cell enriched fractions. The nucleated cell enriched fractions were then subjected to negative selection using the RosetteSep™. Human Mesenchymal Stem Cell Enrichment Cocktail (Stemcell Technologies) followed by density gradient centrifugation or non-physiological conditions followed by density gradient centrifugation to further enrich for fetal nucleated cells. The medium used for non-physiological conditions contained sodium citrate, citric acid, and dextrose, and had an osmolarity of 310-320 mOsm/l.

TABLE 2
Cell recovery from maternal blood subjected to bulk removal
of RBCs and further enrichment for fetal nucleated cells

| Enrichment step | Volume | Expected fetal cells | Observed fetal cells |
|---|---|---|---|
| Starting blood | 25 ml | 25-150 | |
| volume | | | |
| After bulk RBC | 45 ml | 25-150 | |
| removal | | | |
| After negative | 3 ml | 25-150 | 18-250 |
| selection or non- | | | |
| physiological | | | |
| conditions followed | | | |
| by density gradient | | | |
| centrifugation | | | |

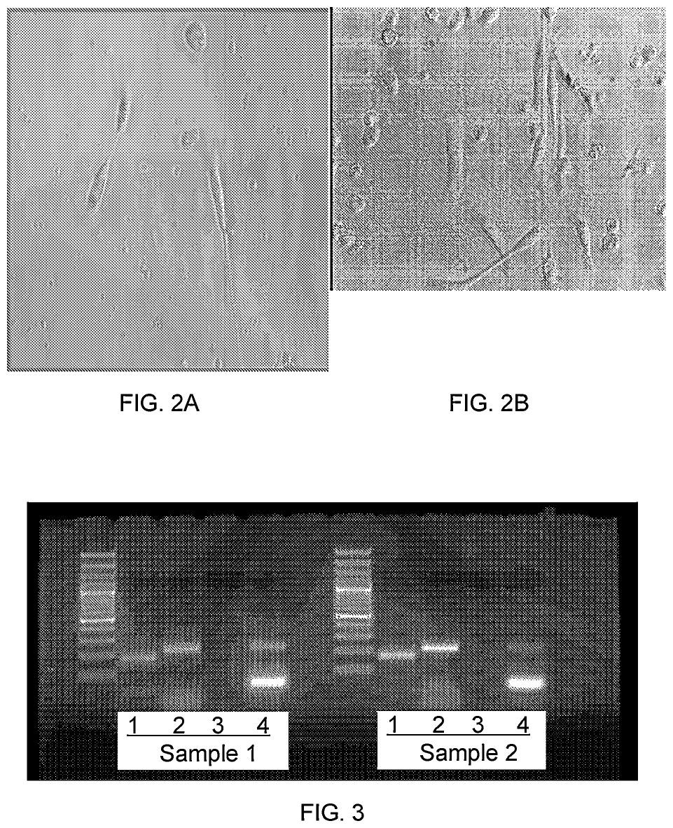

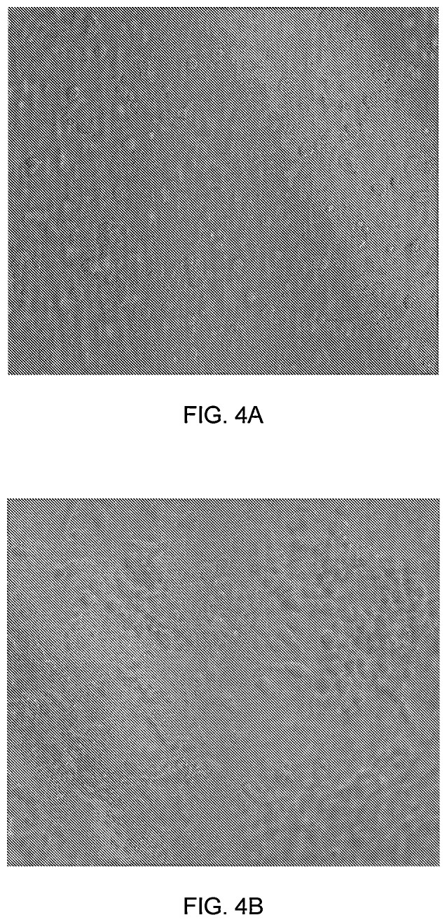

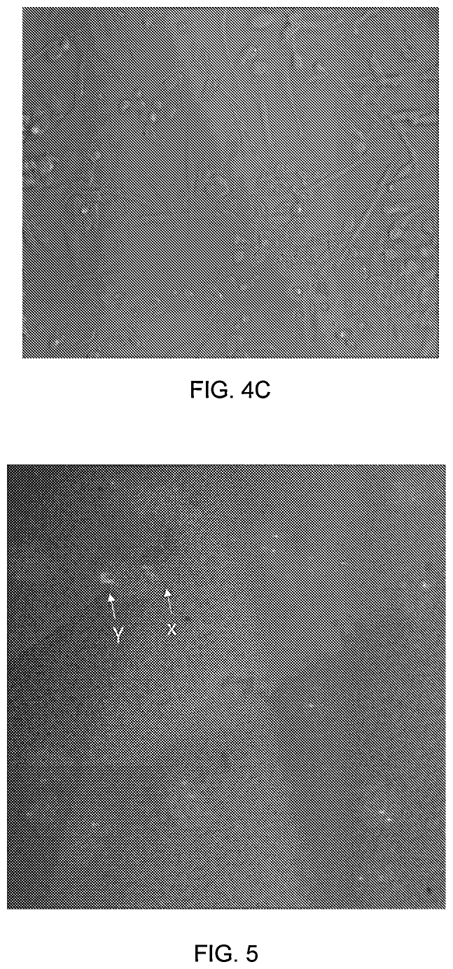

The enriched populations were then cultured in a selective culture medium comprising human stem cell factor (SCF), interleukin 6 (IL-6), FMS-like tyrosine kinase 3 (Flt-3) ligand and megakaryocyte growth and development factor (MGDF) as described in Peters et al., 2010, PLoS ONE 5(12):e15689. Cells cultured for three days and one week are shown in FIG. 2A-2B, respectively. RT-PCR experiments performed on the cells cultured for one week expressed stem cell markers Oct4 and Nanog, as shown in FIG. 3.

### 6.3 Example 3: Target Nucleated Cell Culture and Analysis

A sample of maternal blood from a female 11 week pregnant with twins was processed according to the bulk red blood cell removal protocol of Section 5.5.1 to provide a nucleated cell enriched fraction. Analysis of cell free fetal (cff) DNA present in maternal plasma by RT-PCR with SRY primers and probes had previously determined that at least one of the two fetuses was male. The nucleated cell enriched fraction was then subjected to negative selection using the RosetteSep™ Human Mesenchymal Stem Cell Enrichment Cocktail (Stemcell Technologies) and density gradient centrifugation according to the target nucleated cell enrichment protocol of Section 5.5.2 to select for fetal cells.

The resulting population of cells was then cultured according to the cell culture protocol of Section 5.5.3. At day 3, the cell culture supernatant was collected and fluorescently labeled with a fluorescently labeled anti-CD45 antibody, 4B9, goat anti-mouse IgM Alexa Fluor 488, and DC-Ruby to stain fNRBCs present in the supernatant, and then sorted by FACS into eight tubes with five events/tube. At day 9, ten MSCs were picked by micromanipulation and collected in a single tube. PowerPlex® Fusion short tandem repeat (STR) analysis was performed on the cells sorted by FACS at 3 day and picked by micromanipulation at day 9 to confirm their fetal identities. The remaining sample at day 9 was analyzed by FISH with X and Y chromosome specific probes.

Paternal alleles were identified in the FACS sorted cells in the cells picked by micromanipulation (data not shown). X and Y chromosomes were observed by FISH, indicating the presence of male fetal cells (see FIG. 5).

### 7. SPECIFIC EMBODIMENTS

The present disclosure is exemplified by the specific embodiments below.

1. A process for isolating a population of target nucleated cells from non-nucleated red blood cells present in a blood derived nucleated cell enriched fraction containing no more than 20% of the non-nucleated red blood cells from the blood, comprising subjecting the nucleated cell enriched fraction to at least one of:
a) negative selection for the target nucleated cells;
b) positive selection for the target nucleated cells; and
c) density gradient centrifugation,
   thereby isolating a population of target nucleated cells.
   2. The process of embodiment 1, wherein the nucleated cell enriched fraction contains at least 85%, at least 90%, at least 95%, or at least 99% of the nucleated cells from the blood.
   3. The process of embodiment 1 or embodiment 2, wherein the nucleated cell enriched fraction contains no more than 15%, no more than 10%, no more than 5%, or no more than 1% of the non-nucleated red blood cells from the blood.
   4. The process of any one of embodiments 1 to 3, wherein the nucleated cell enriched fraction is the product of a process comprising:
a) separating a mixture comprising nucleated cells, non-nucleated red blood cells, and an aggregating agent into a nucleated cell enriched fraction and a non-nucleated red blood cell enriched fraction in a lumen of a container at local gravity, wherein the separating is performed in batch, and wherein
   i. the average height of the mixture in the lumen is no more than 4 cm; and/or
   ii. the average height of the mixture in the lumen is selected to provide a non-nucleated red blood cell enriched fraction that contains at least 80% of the non-nucleated red blood cells in the mixture and/or no more than 20% of the nucleated cells in the mixture after no more than 3 rounds, no more than 2 rounds or no more than one round of separation;
b) optionally repeating step (a) one or more times; and
c) optionally washing the nucleated cell enriched fraction one or more times.
   5. The process of embodiment 4, wherein the washing comprises concentrating the cells of the nucleated cell enriched fraction and combining the concentrated cells with a solution comprising plasma.
   6. The process of embodiment 5, in which the plasma is autologous plasma.
   7. The process of any one of embodiments 4 to 6, wherein the average height of the mixture in the lumen is no more than 4 cm, no more than 3.5 cm, no more than 3 cm, no more than 2.5 cm, no more than 2 cm, or no more than 1.5 cm.
   8. The process of any one of embodiments 4 to 6, wherein the average height of the mixture in the lumen is no more than 1 cm.
   9. The process of any one of embodiments 4 to 8, wherein the average height of the mixture in the lumen is at least 0.5 cm or at least 1 cm.
   10. The process of any one of embodiments 4 to 9, wherein the volume of the mixture is less than 500 mL, less than 400 mL, less than 300 mL, less than 200 mL, less than 100 mL, less than 75 mL, less than 50 mL, less than 40 mL, less than 30 mL, or less than 25 mL.
   11. The process of any one of embodiments 4 to 10, wherein the volume of the mixture is at least 5 mL, at least 10 mL, at least 20 mL or at least 25 mL.
   12. The process of any one of embodiments 4 to 9, wherein the volume of the mixture is 25 mL to 50 mL, 50 mL to 100 mL, 100 mL to 200 mL, or 200 mL to 400 mL.
   13. The process of any one of embodiments 4 to 12, wherein the aggregating agent is dextran, hydroxyethyl starch (HES), gelatin, pentastarch, ficoll, gum ararbic, polyvinylpyrrolidone, 5-(N-2,3-dihydroxypropylacetamido)-2,4,6-tri-iodo-N,N′-bis (2,3 dihydroxypropyl) isophthalamide or any combination thereof.
   14. The process of any one of embodiments 4 to 13, wherein the separating is for 2 to 15 minutes, 2 to 10 minutes, 2 to 5 minutes, 3 to 6 minutes, 4 to 12 minutes, 5 to 10 minutes, 2 to 8 minutes, 4 to 10 minutes, 3 to 7 minutes, 6 to 10 minutes, or 5 to 8 minutes.
   15. The process of any one of embodiments 4 to 14, wherein the lumen of the container has a fixed volume.
   16. The process of any one of embodiments 4 to 15, wherein the lumen of the container comprises a cylindrical section or a polyhedral section.
   17. The process of any one of embodiments 4 to 16, wherein the lumen of the container comprises a funnel shaped section.
   18. The process of any one of embodiments 4 to 16, wherein the lumen of the container comprises a cylindrical section or polyhedral section joined (a) at the bottom end to a funnel shaped section, (b) at one the top end to an inverted funnel shaped section, or (c) at the bottom end to a funnel shaped section and at the top end to an inverted funnel shaped section.
   19. The process of any one of embodiments 4 to 18, wherein container comprises one or more inlet/outlet ports operably connected to the lumen of the container.
   20. The process of embodiment 19, wherein the container further comprises one or more flow deflectors positioned within the lumen of the container to allow for the deflection of a fluid introduced into the lumen of the container through the one or more of the inlet/outlet ports and to provide a laminar fluid flow.
   21. The process of embodiment 19 or embodiment 20, wherein the process for producing the nucleated cell enriched fraction further comprises introducing a heavy liquid into the lumen of the container through a first inlet/outlet port positioned at the bottom of the lumen until all or part of the nucleated cell enriched fraction is forced out of the lumen through a second inlet/outlet port positioned at the top of the lumen.
   22. The process of embodiment 21, wherein an amount of the heavy liquid is present in the lumen of the container during the separation of the mixture.
   23. The process of embodiment 21 or embodiment 22, wherein the heavy liquid comprises heptacosafluorotributylamine, Ficoll 1.077 g/mL, Ficoll 1.085 g/mL, or any combination thereof.
   24. The process of embodiment 22 or embodiment 23, wherein in the process for producing the nucleated cell enriched fraction, the mixture is introduced into the lumen of the container after the amount of the heavy liquid is introduced into the container.
   25. The process of embodiment 24, wherein the process for producing the nucleated cell enriched fraction comprises a step of introducing the amount of the heavy liquid into the container before introducing the mixture into the lumen of the container.
   26. The process of any one of embodiments 4 to 25, wherein the process for producing the nucleated cell enriched fraction comprises a step of introducing the mixture into the lumen of the container.
   27. The process of any one of embodiments 4 to 26, wherein the mixture is the product of a process comprising combining the aggregating agent or a solution comprising the aggregating agent and a sample comprising the nucleated cells and the non-nucleated red blood cells.
   28. The process of embodiment 27, wherein the process for producing the nucleated cell enriched fraction further comprises a step of forming the mixture.
   29. The process of embodiment 27 or embodiment 28, wherein the sample is a previously prepared nucleated cell enriched fraction.
   30. The process of embodiment 27 or embodiment 28, wherein the sample comprises whole blood.
   31. The process of embodiment 27 or embodiment 28, wherein the sample comprises a blood fraction.
   32. The process of embodiment 31, wherein the blood fraction contains at least 5%, at least 10%, at least 20%, at least 30%, at least 40%, at least 50%, or more than 50% of the plasma present in an amount of whole blood used to make the blood fraction.
   33. The process of embodiment 31, wherein the blood fraction contains 5-10%, 10-20%, 20-30%, 20-50%, or 50-100% of the plasma present in an amount of whole blood used to make the blood fraction.
   34. The process of any one of embodiments 31 to 33, wherein the blood fraction is the product of a process comprising:
a) optionally, diluting blood with an aqueous solution;
b) centrifuging blood or the diluted blood from step (a) to obtain a cell pellet; and
c) optionally, resuspending the pellet in an aqueous solution, which aqueous solution has the same composition as the aqueous solution of step (a) or has a different composition from the aqueous solution of step (a),
thereby forming the blood fraction.
   35. The process of embodiment 27 or embodiment 28, wherein the sample comprises blood diluted with an aqueous solution.
   36. The process of any one of embodiments 34 to 35, wherein the aqueous solution comprises plasma, a cell culture medium, a buffered solution, or a combination thereof.
   37. The process of embodiment 36, wherein the cell culture medium is Roswell Park Memorial Institute (RPMI) medium, Earle medium, or Hanks balanced salt solution.
   38. The process of any one of embodiments 4 to 37, wherein the mixture is isotonic to red blood cells.
   39. The process of any one of embodiments 4 to 38, further comprising a step of producing the nucleated cell enriched fraction.
   40. The process of any one of embodiments 1 to 39, wherein the negative selection comprises negative immunoselection.
   41. The process of any one of embodiments 1 to 40, wherein the positive selection comprises positive immunoselection.
   42. The process of any one of embodiments 1 to 41, which comprises subjecting the nucleated cell enriched fraction to negative selection followed by density centrifugation.
   43. The process of any one of embodiments 1 to 42, wherein the density gradient centrifugation is preceded by a step of incubating the nucleated cell enriched fraction in a medium having non-physiological conditions.
   44. The process of any one of embodiments 1 to 43, wherein the blood is peripheral blood or umbilical cord blood.
   45. The process of embodiment 44, wherein the blood is peripheral blood from a pregnant female, a subject afflicted with a cancer, or blood obtained from a healthy individual.
   46. The process of embodiment 45, wherein blood is peripheral blood from a pregnant female and the target nucleated cells comprise fetal cells.
   47. The process of any one of embodiments 1 to 46 wherein the target nucleated cells comprise rare nucleated cells.
   48. The process of embodiment 47, wherein the rare nucleated cells comprise stem cells or cancer cells.
   49. The process of embodiment 48, wherein the stem cells comprise mesenchymal stem cells.
   50. The process of embodiment 49, wherein the negative selection comprises negative immunoselection utilizing one or more antibodies against one or more cell surface markers, wherein at least one of the cell surface markers is selected from CD2, CD3, CD10, CD11b, CD14, CD15, CD16, CD19, CD31, CD34, CD35, CD38, CD44, CD45, CD49, CD49d, CD56, CD61, CD62(E), CD66b, CD68, CD79alpha, CD104, CD106, CD117, HLA-DR, and glycophorin A.
   51. The process of embodiment 50, wherein:
a) the negative immunoselection utilizes one or more antibodies against one or more cell surface markers, wherein at least one of the cell surface markers is selected from CD3, CD14, CD19, CD38, CD66b, and glycophorin A;
b) the negative immunoselection utilizes one or more antibodies against two or more cell surface markers, wherein at least two of the cell surface markers are selected from CD3, CD14, CD19, CD38, CD66b, and glycophorin A;
c) the negative immunoselection utilizes one or more antibodies against three or more cell surface markers, wherein at least three of the cell surface markers are selected from CD3, CD14, CD19, CD38, CD66b, and glycophorin A;
d) the negative immunoselection utilizes one or more antibodies against four or more cell surface markers, wherein at least four of the cell surface markers are selected from CD3, CD14, CD19, CD38, CD66b, and glycophorin A;
e) the negative immunoselection utilizes one or more antibodies against five or more cell surface markers, wherein at least five of the cell surface markers are selected from CD3, CD14, CD19, CD38, CD66b, and glycophorin A; or
a) the negative immunoselection utilizes one or more antibodies against six or more cell surface markers, wherein at least six of the cell surface markers are selected from CD3, CD14, CD19, CD38, CD66b, and glycophorin A.
   52. The process of any one of embodiments 49 to 51, wherein the positive selection comprises positive immunoselection utilizing one or more antibodies against one or more cell surface markers, wherein at least one of the cell surface markers is selected from CD73, CD90, CD105, CD166, CD200, CD271, and STRO-1.
   53. The process of any one of embodiments 1 to 52, further comprising culturing the population of target nucleated cells.
   54. The process of embodiment 53, wherein the target nucleated cells comprise mesenchymal stem cells and culturing comprises culturing the population of target nucleated cells in a mesenchymal stem cell medium.
   55. A population of target nucleated cells obtained by the process of any one of embodiments 1 to 54.
   56. The population of target nucleated cells of embodiment 55 which comprises fetal mesenchymal stem cells.
   57. The population of embodiment 56, wherein at least 70%, at least 80%, at least 90%, or at least 95% of the cells in the population are fetal mesenchymal stem cells.
   58. A method of detecting a fetal abnormality, comprising analyzing at least one fetal mesenchymal stem cell from the population of embodiment 56 or embodiment 57 for a fetal abnormality.
   59. The method of embodiment 58, which comprises analyzing a single mesenchymal stem cell for the fetal abnormality.
   60. The method of embodiment 58, which comprises analyzing a group of mesenchymal stem cells for the fetal abnormality.
   61. The method of embodiment 59 or embodiment 60 which comprises performing whole genome amplification prior to said analyzing.
   62. The method of embodiment 59 or embodiment 60, which comprises amplifying a subset of the genome prior to said analyzing.
   63. The method of any one of embodiments 58 to 62, wherein the analysis comprises quantitative PCR.
   64. The method of any one of embodiments 58 to 63, wherein the analysis is performed on a microarray.
   65. The method of any one of embodiments 58 to 64, wherein the analysis comprises fluorescence in situ hybridization (FISH).
   66. The method of any one of embodiments 58 to 65, which further comprises validating the mesenchymal stem cell or mesenchymal stem cells as fetal cells.
   67. The method of embodiment 66, wherein validation comprises performing fluorescence in situ hybridization (FISH), short tandem repeat (STR) analysis, genetic fingerprinting, single nucleotide polymorphism (SNP) analysis or any combination thereof.
   68. The method of embodiment 66 or embodiment 67, wherein validation comprises comparing mesenchymal stem cell DNA to maternal DNA.
   69. The method of embodiment 66 or embodiment 67, wherein validation comprises comparing mesenchymal stem cell DNA to both maternal and paternal DNA.
   70. A method for in utero stem cell therapy, comprising delivering a population of target nucleated cells according to embodiment 56 or embodiment 57 to a fetus in utero.
   71. The method of embodiment 70, wherein the fetus has a neural tube defect.
   72. The method of embodiment 70, wherein the fetus has a gene defect that causes a disease or disorder.
   73. The method of embodiment 72, wherein the disease or disorder is a hematological disease, a metabolic disease, an immunological disease, or a bone disorder.
   74. The method of embodiment 72 or embodiment 73, wherein the target nucleated cells comprise mesenchymal stem cells comprising a gene of which the fetus is in need.
   75. The method of embodiment 74, wherein the mesenchymal stem cells comprise allogeneic mesenchymal stem cells.
   76. The method of embodiment 74, wherein the mesenchymal stem cells comprise autologous mesenchymal stem cells made recombinant by introduction of the gene of which the fetus is in need.
   77. A method of treating a subject afflicted with a disease or condition, comprising administering a population of target nucleated cells according to embodiment 56 or embodiment 57 to the subject, wherein the disease or condition is selected is from a wound, an orthopedic injury, a cardiovascular disease, an autoimmune disease, a liver disease, a neurological disorder, neuronal degeneration, graft versus host disease, a metabolic disease, renal infarction, and myocardial infarction.
   78. A method of promoting hematopoietic cell engraftment in a subject receiving a hematopoietic stem cell transplant, comprising administering a population of target nucleated cells according to embodiment 56 or embodiment 57 to the subject.
   79. A method of regenerating bone and/or cartilage in a subject in need thereof, comprising administering a population of target nucleated cells according to embodiment 56 or embodiment 57 to the subject.

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A process for isolating a population of target nucleated cells from non-nucleated red blood cells present in a blood derived nucleated cell enriched fraction containing no more than 20% of the non-nucleated red blood cells from the blood, comprising subjecting the nucleated cell enriched fraction to at least one of:
a) negative selection for the target nucleated cells;
b) positive selection for the target nucleated cells; and
c) density gradient centrifugation,
thereby isolating a population of target nucleated cells.

2. The process of claim 1, wherein the nucleated cell enriched fraction contains:
a) at least 85%, at least 90%, at least 95%, or at least 99% of the nucleated cells from the blood; and/or
b) no more than 15%, no more than 10%, no more than 5%, or no more than 1% of the non-nucleated red blood cells from the blood.

3. The process of claim 1, wherein the nucleated cell enriched fraction is the product of a process comprising:
a) separating a mixture comprising nucleated cells, non-nucleated red blood cells, and an aggregating agent into a nucleated cell enriched fraction and a non-nucleated red blood cell enriched fraction in a lumen of a container at local gravity, wherein the separating is performed in batch, and wherein
i. the average height of the mixture in the lumen is no more than 4 cm;
ii. the average height of the mixture in the lumen is selected to provide a non-nucleated red blood cell enriched fraction that contains at least 80% of the non-nucleated red blood cells in the mixture and/or no more than 20% of the nucleated cells in the mixture after no more than 3 rounds, no more than 2 rounds or no more than one round of separation;
b) optionally repeating step (a) one or more times; and
c) optionally washing the nucleated cell enriched fraction one or more times.

4. (canceled)

5. The process of claim 3, wherein the average height of the mixture in the lumen is:
a) no more than 4 cm, no more than 3.5 cm, no more than 3 cm, no more than 2.5 cm, no more than 2 cm, no more than 1.5 cm, or no more than 1 cm; and/or
b) at least 0.5 cm or at least 1 cm.

6. The process of claim 3, wherein the volume of the mixture is:
a) less than 500 mL, less than 400 mL, less than 300 mL, less than 200 mL, less than 100 mL, less than 75 mL, less than 50 mL, less than 40 mL, less than 30 mL, or less than 25 mL;
b) at least 5 mL, at least 10 mL, at least 20 mL or at least 25 mL;
c) any combination of a) and b); or
d) 25 mL to 50 mL, 50 mL to 100 mL, 100 mL to 200 mL, or 200 mL to 400 mL.

7. The process of claim 3, wherein the aggregating agent is dextran, hydroxyethyl starch (HES), gelatin, pentastarch, ficoll, gum ararbic, polyvinylpyrrolidone, 5-(N-2,3-dihydroxypropylacetamido)-2,4,6-tri-iodo-N,N′-bis (2,3 dihydroxypropyl) isophthalamide or any combination thereof.

8. (canceled)

9. The process of claim 3, wherein the lumen of the container:
a) has a fixed volume;
b) comprises a funnel shaped section;
c) comprises a cylindrical section or a polyhedral section, optionally wherein the cylindrical section or polyhedral section is joined:
(i) at the bottom end to a funnel shaped section;
(ii) at one the top end to an inverted funnel shaped section; or
(iii) at the bottom end to a funnel shaped section and at the top end to an inverted funnel shaped section;
or
d) any combination of a)-c).

10. (canceled)

11. (canceled)

12. (canceled)

13. The process of claim 3, wherein the mixture is the product of a process comprising combining the aggregating agent or a solution comprising the aggregating agent and a sample comprising the nucleated cells and the non-nucleated red blood cells, optionally wherein the process for producing the nucleated cell enriched fraction further comprises a step of forming the mixture.

14. The process of claim 13, wherein the sample:
a) is a previously prepared nucleated cell enriched fraction;
b) comprises whole blood;
c) comprises a blood fraction, optionally wherein
i) A) the blood fraction contains at least 5%, at least 10%, at least 20%, at least 30%, at least 40%, at least 50%, or more than 50% of the plasma present in an amount of whole blood used to make the blood fraction; or
B) the blood fraction contains 5-10%, 10-20%, 20-30%, 20-50%, or 50-100% of the plasma present in an amount of whole blood used to make the blood fraction; and/or
ii) the blood fraction is the product of a process comprising:
(1) optionally, diluting blood with an aqueous solution;
(2) centrifuging blood or the diluted blood from step (1) to obtain a cell pellet; and
(3) optionally, resuspending the pellet in an aqueous solution, which aqueous solution has the same composition as the aqueous solution of step (1) or has a different composition from the aqueous solution of step (1),
thereby forming the blood fraction; or
d) comprises blood diluted with an aqueous solution.

15. (canceled)

16. (canceled)

17. The process of claim 1, wherein:
a) the negative selection comprises negative immunoselection;
b) the positive selection comprises positive immunoselection;
c) the process comprises subjecting the nucleated cell enriched fraction to negative selection followed by density centrifugation;
d) the density gradient centrifugation is preceded by a step of incubating the nucleated cell enriched fraction in a medium having non-physiological conditions;
e) the blood is peripheral blood or umbilical cord blood, optionally wherein the blood is peripheral blood from a pregnant female, a subject afflicted with a cancer, or blood obtained from a healthy individual, optionally wherein the blood is peripheral blood from a pregnant female and the target nucleated cells comprise fetal cells;
f) the target nucleated cells comprise rare nucleated cells, optionally wherein the rare nucleated cells comprise stem cells or cancer cells, optionally wherein the stem cells comprise mesenchymal stem cells; or
g) any combination of a)-f).

18. The process of claim 17, wherein the target nucleated cells comprise mesenchymal stem cells and the negative selection comprises negative immunoselection utilizing one or more antibodies against one or more cell surface markers, wherein at least one of the cell surface markers is selected from CD2, CD3, CD10, CD11b, CD14, CD15, CD16, CD19, CD31, CD34, CD35, CD38, CD44, CD45, CD49, CD49d, CD56, CD61, CD62(E), CD66b, CD68, CD79alpha, CD104, CD106, CD117, HLA-DR, and glycophorin A.

19. The process of claim 18, wherein:
a) the negative immunoselection utilizes one or more antibodies against one or more cell surface markers, wherein at least one of the cell surface markers is selected from CD3, CD14, CD19, CD38, CD66b, and glycophorin A;
b) the negative immunoselection utilizes one or more antibodies against two or more cell surface markers, wherein at least two of the cell surface markers are selected from CD3, CD14, CD19, CD38, CD66b, and glycophorin A;
c) the negative immunoselection utilizes one or more antibodies against three or more cell surface markers, wherein at least three of the cell surface markers are selected from CD3, CD14, CD19, CD38, CD66b, and glycophorin A;
d) the negative immunoselection utilizes one or more antibodies against four or more cell surface markers, wherein at least four of the cell surface markers are selected from CD3, CD14, CD19, CD38, CD66b, and glycophorin A;
e) the negative immunoselection utilizes one or more antibodies against five or more cell surface markers, wherein at least five of the cell surface markers are selected from CD3, CD14, CD19, CD38, CD66b, and glycophorin A; or
f) the negative immunoselection utilizes one or more antibodies against six or more cell surface markers, wherein at least six of the cell surface markers are selected from CD3, CD14, CD19, CD38, CD66b, and glycophorin A.

20. The process of claim 17, wherein the target nucleated cells comprise mesenchymal stem cells and wherein the positive selection comprises positive immunoselection utilizing one or more antibodies against one or more cell surface markers, wherein at least one of the cell surface markers is selected from CD73, CD90, CD105, CD166, CD200, CD271, and STRO-1.

21. (canceled)

22. A population of target nucleated cells obtained by the process of claim 1, which optionally comprises fetal mesenchymal stem cells, optionally wherein at least 70%, at least 80%, at least 90%, or at least 95% of the cells in the population are fetal mesenchymal stem cells.

23. A method of detecting a fetal abnormality, comprising analyzing at least one fetal mesenchymal stem cell from a population of claim 22 which comprises mesenchymal stem cells for a fetal abnormality, optionally wherein the method comprises:
a) analyzing a single mesenchymal stem cell for the fetal abnormality; or
b) analyzing a group of mesenchymal stem cells for the fetal abnormality, optionally wherein the method comprises:
i) performing whole genome amplification prior to said analyzing; or
ii) amplifying a subset of the genome prior to said analyzing.

24. (canceled)

25. The method of claim 23, which further comprises validating the mesenchymal stem cell or mesenchymal stem cells as fetal cells, optionally wherein validation comprises performing fluorescence in situ hybridization (FISH), short tandem repeat (STR) analysis, genetic fingerprinting, single nucleotide polymorphism (SNP) analysis or any combination thereof.

26. (canceled)

27. A method for in utero stem cell therapy, comprising delivering a population of target nucleated cells according to claim 22 which comprises fetal mesenchymal stem cells to a fetus in utero, optionally wherein the fetus:
a) has a neural tube defect; or
b) has a gene defect that causes a disease or disorder, optionally wherein:
i) the disease or disorder is a hematological disease, a metabolic disease, an immunological disease, or a bone disorder; and/or
ii) the target nucleated cells comprise mesenchymal stem cells comprising a gene of which the fetus is in need, optionally wherein the mesenchymal stem cells comprise:
A) allogeneic mesenchymal stem cells; or
B) autologous mesenchymal stem cells made recombinant by introduction of the gene of which the fetus is in need.

28. A method of treating a subject afflicted with a disease or condition, comprising administering a population of target nucleated cells according to claim 22 which comprises fetal mesenchymal stem cells to the subject, wherein the disease or condition is selected is from a wound, an orthopedic injury, a cardiovascular disease, an autoimmune disease, a liver disease, a neurological disorder, neuronal degeneration, graft versus host disease, a metabolic disease, renal infarction, and myocardial infarction.

29. A method of promoting hematopoietic cell engraftment in a subject receiving a hematopoietic stem cell transplant, comprising administering a population of target nucleated cells according to claim 22 which comprises fetal mesenchymal stem cells to the subject.

30. A method of regenerating bone and/or cartilage in a subject in need thereof, comprising administering a population of target nucleated cells according to claim 22 which comprises fetal mesenchymal stem cells to the subject.
